(19) United States
(12) Patent Application Publication
(10) Pub. No.: US 2024/0038374 A1
(43) Pub. Date: Feb. 1, 2024

(54) SYSTEM AND METHOD FOR MEDICAL PRACTICE ANALYSIS AND MANAGEMENT

(71) Applicants: Sean Patrick Miller, Kennebunk, ME (US); Biswa Prakash, Raymond, ME (US); William Joseph Griffin, Edgewater, MD (US); Jeremy James Halperin, Portland, ME (US); Daniel Eugenio Espinal, Cape Elizabeth, ME (US); Brent Philip Profenno, South Portland, ME (US)

(72) Inventors: Sean Patrick Miller, Kennebunk, ME (US); Biswa Prakash, Raymond, ME (US); William Joseph Griffin, Edgewater, MD (US); Jeremy James Halperin, Portland, ME (US); Daniel Eugenio Espinal, Cape Elizabeth, ME (US); Brent Philip Profenno, South Portland, ME (US)

(21) Appl. No.: 18/359,445

(22) Filed: Jul. 26, 2023

Related U.S. Application Data
(60) Provisional application No. 63/392,680, filed on Jul. 27, 2022.

(51) Int. Cl. G16H 40/20

(52) U.S. Cl. CPC G16H 40/20

(57) ABSTRACT

A system and platform are provided that enables end-users to gain insight into group and individual practices. Those insights may be obtained for any desirable aspect of each practice and the collection of practices including, but not limited to, financial information, medical information, human resources information, and patient information. That information can be used by group management to identify best practices, and it can be used to advise individual practices on strengths to be reinforced and weaknesses to be addressed. That information may be conveyed to group management and individual practices in the form of dashboards customizable to the particular functions of specific users and their personas.

### RELATED APPLICATIONS

This application claims the benefit under 35 U.S.C. § 119(e) of U.S. provisional patent application Ser. No. 63/392,680 entitled “SYSTEM AND METHOD FOR MEDICAL PRACTICE ANALYSIS AND MANAGEMENT,” filed Jul. 27, 2022, Attorney Docket No. R0866.70000US00, which is herein incorporated by reference in its entirety.

### SUMMARY

Various aspects as described herein relate generally to a software and hardware platform, and human platform, and related methods for gathering information related to aspects of a collection of practices and core internal platforms, analyzing that information and providing managerial, operational, and clinical feedback associated with one or more practices of the collection to front line teams. Such practices include, for example, veterinary and medical practices. The data gathering and analytics begins at the pre-acquisition phase, which serves as the foundational metrics to improve operational performance and to accelerate the practice integration into our network.

More specifically, various embodiments relate to a software, hardware and human architecture and ecosystem that may be used across multiple agnostic practice software ecosystems, and core enterprise systems to normalize, analyze, visualize, and implement data driven business and clinical decisions to end-users. The end-product presents accurate, actionable, operational and clinical data to help teams in near-real time improve financial, medical, managerial and end-user experience. Further, the platform includes people analytics to improve business and medical outcomes provided by service providers. Such systems may be applicable, for example, such as service industries including veterinary and medical practices, but may also be used in other practice areas (e.g., consulting, professional services, legal services, etc.), where service performance, financials and client service may be evaluated. However, some embodiments are particularly applicable in the veterinary and medical practice areas, especially in multi-site low regulation environments where multiple practices (e.g., veterinary practices, hospitals, etc.) need to be evaluated and monitored with low oversight.

Traditionally, managers of individual practices (e.g., veterinary practices) have available to them an array of management tools, most often in the form of computer-based applications, for various functions. The most common applications include but are not limited to those that aid financial management of the practice, clinical content, client and patient information and management, and human resources. There is wide availability of such applications and there is little if any standardization among them. In addition, there is little regulation of the business and clinical processes of veterinary practices. It is therefore common in the field of veterinary practices to have ad hoc platforms with different application components that are not compatible, and have disparate data sources.

As a result, the inventors appreciate that each practice tends to be unique in the larger sense of how it operates, metrics defined (if any) for desired performance of the business, and management insight into what works and what does not. Not surprisingly then, some practices perform better than others from efficiency, profitability, client retention, employee retention, quality of medicine and satisfaction. For those practices seeking to improve in any one or more of those areas, it is not unusual to look to changes in one or more computer-based management tools. Once the management tools are established and employees trained to use them, it can be very difficult to make changes, whether for interoperability of new and existing programs, or employee training and buy-in to make a transition. Any attempt to make improvements is layered onto the ongoing difficulties and complexities associated with day-to-day operations of the business.

The complexities of veterinary practice management described are magnified substantially for the business engaged in combining and operating multiple practices into a single entity. It is understood that there can be benefits to the practices in such combinations, such as backend management activities that are ancillary but important to the primary function of patient care. It is also understood that for the aggregator or multi-site operator, there can be financial advantages to managing those practices with their established patient bases. However, with the disparate platforms and programs employed by existing practices, unintegrated and not standardized, the aggregator and multi-site hospital operator expends substantial energy and resources bringing consistency and, hopefully, efficiencies to the group of practices. It is desirable to minimize the aggravations associated with combining veterinary practices.

Those aggravations resulting from the changes that occur when a veterinary practice is acquired can be disruptive in both directions. If an aggregator seeks to normalize all practices in the group, there will be a change in platforms, programs, or both used by the practices. Those changes can be difficult and time-consuming to implement. They can also lead to upheaval in the practice as employees either struggle to learn new systems while continuing with patient care, or they simply leave the practice to avoid the aggravation. These disruptions can also lead to confusion and suboptimal outcomes for clients and patients as in many cases they require client adoption. Should the aggregator decide to avoid those aggravations to the practices, efficiencies of the combination can be lost, with substantial time spent addressing problems across multiple platforms and losing insight into features of the practices of importance to group management as well as obscurity in patient data. Analysis of practice activities can be difficult and incomplete.

Therefore, what is needed is a system and method to enable the efficient analysis, deliver it to the end-users, and enable self-management of a group of practices (e.g., a veterinary, medical or other type of practice) based on key predictive indices. Further, what is needed is such a system and method configured to minimize disruption of the ordinary activities of the practices of the group, while also configured to maximize insight into the business and clinical operations of each practice and streamline decision making at all levels of the organization. Yet further, what is needed is such a system and method configured to aggregate practice information in an efficient manner, analyze that information by individual practice and the group. Also, it is desirable to have the system and method configured to be able to return to one or more practices of the group insights into operational, medical, managerial, and end-user insights efficiencies identified through the information gathering and analysis processes.

In some embodiments, such information is developed without disturbing existing practice management solutions, and in some architectures, the system interfaces with existing practice management components to extract and normalize data. Further, in some embodiments, the system is capable of interfacing with multiple practices for the purpose of management and/or evaluating potential of acquisition (e.g., by an aggregator of practices). The system architecture may be implemented as one or more microservices operating in a SaaS (Software as a Service) architecture. In some embodiments, the system may comprise one or more interface components for receiving information from the conventional practice management systems, and when configured, may obtain real-time performance data (e.g., data and events) from these conventional systems. The system may also provide one or management interfaces (e.g., management dashboards, operations dashboards, practice dashboards, etc.) to permit various user types to manage the practice more efficiently in near real time.

In some embodiments, such an architecture may be configured to, after incorporation by an aggregator or multi-site operator, to receive real-time practice management information for the purpose of determining certain performance indicators associated with the practice. Such indicators may be, for example, financial, behavioral, patient-related, client-related or may relate to the actual practice and treatments (e.g., medically related indicators). In some embodiments, the system may also store and track the quality of patient care over time and may be configured to make recommendations regarding patient care to improve the patient outcome based on attributes such as species, breed, age, medical history, etc. The system, having deep and consistent visibility of multiple practices, may make insightful recommendations for a particular patient that differentiate solutions based on needs of the patient, such as urgent care, general care, and specialty services. Further, the system may provide early indicators into a decision-making matrix for veterinary medicine or other healthcare environments with high-accessibility to data and limited regulatory constraint.

In some embodiments, a system and method are provided to enable the efficient analysis and management of a group of practices (e.g., veterinary practices). It is also an object to provide a system and method to minimize disruption of the ordinary activities of the practices of the group, while maximizing insight into the business of each practice, as well as optimizing medical outcomes. Further, it is an object to enable practice information aggregation in an efficient manner, and analysis of that information by individual practice and the group. The system and method should be configured to enable return to one or more practices of the group insights into operational, medical, and end-user efficiencies identified through the information gathering and analysis processes. Further, the system may be configured to provide the client with early indicators about the patient into a decision-making matrix applied to veterinary medicine.

The software applications can also be configured to harvest data for analysis that enables end-users to gain insight into the group and individual practices, program. In some embodiments, the system is configured to aggregate data collected from practice management systems merged with corporate systems into a format that permits the data to be analyzed to permit insights into the practice. Those insights may be obtained for any desirable aspect of each practice and the collection of practices including, but not limited to, financial, medical, behavioral, and patient information. That information can be used by group management to identify best practices, and it can be used to advise individual practices on strengths to be reinforced and weaknesses to be addressed. That information may be conveyed to group management and individual practices in the form of dashboards customizable to the functions of specific end-users and their personas.

The one or more computer programs define a system architecture that allows for onboarding of any information of interest, regardless of particular format of that information. Individual practices are therefore not required to adopt specific new or different platforms and/or applications for the benefit of group management. In some embodiments, the system is agnostic with respect to individual practice system usage, and disparate internal core systems. This shifts a substantial portion of the burden of the transition of an individual practice into a practice group from that practice and group management onto the system according to various embodiments. A database of the collected data is used to store that data in a metadata configuration as an aspect of data analysis. This enable the platform to be interoperable with all systems within the ecosystem.

The system and method according to some embodiments enable an efficient mechanism for gaining insight into individual practices and the aggregation of those practices into a group. That is accomplished with a mechanism for effective gathering of relevant data from a practice regardless of the platforms and applications used by that practice. The system is further configured to characterize and analyze that gathered data, feed some or all of the analysis back to the practice and/or the group of practices and provide group management with insight into the performance of individual practices and their employees, the group as a whole and best practices for veterinary activities. The information gathered and analyzed may be viewed by relevant system users in an understandable configuration, such as a dashboard, for example.

According to one aspect is a distributed computer system is provided that permits management of practice data. The system comprises a component configured to extract practice management data from one or more practice management data sources relating to a practice being evaluated, a component configured to map the extracted practice management data into at least one normalized data set, a component configured to process and store transactional data from the extracted practice management data, and a component adapted to analyze the normalized data set and transactional data; and responsive to an analysis operation, determine a performance measure of the practice being evaluated.

According to one embodiment, one or more practice management data sources relating to the practice being evaluated comprise one or more of a group including financial information, human resource information, behavioral information, patient information, and medical information. According to one embodiment, the component adapted to analyze the normalized data set and transactional data; and responsive to an analysis operation, determine the performance measure of the practice being evaluated determines a performance measure related to at least one of a group comprising financial performance, human resource performance, behavioral performance, patient performance, and medical performance. According to one embodiment, the system comprises an interface component configured to output the performance measure related to at least one of the group comprising financial performance, human resource performance, behavioral performance, patient performance, and medical performance to at least one user type or computing entity.

According to one embodiment, at least one user type comprises at least one of a group of user types including a practice management user, an outside evaluator user evaluating the practice being evaluated, and an employee user of the practice being evaluated. According to one embodiment, the system comprises a plurality of components that interface with existing ones or the one or more practice management data sources. According to one embodiment, the performance measure of the practice being evaluated further comprises at least one of a group including an employee experience index, a client experience index, a financial index, a labor index, an inventory index, a volume index, and a revenue index. According to one embodiment, the performance measure of the practice being evaluated further comprises at least one of a group including root cause, plan information, and progress toward a target.

According to one embodiment, the system comprises a component configured to determine a performance measure across multiple practices. According to one embodiment, the system comprises a component configured to display the performance measure of the practice. According to one embodiment, the displayed performance measure may be visualized using at least one of a group including a chart, plot, or table.

According to one aspect, a method for managing practice data is provided. The method comprises extracting practice management data from one or more practice management data sources relating to a practice being evaluated, mapping the extracted practice management data into at least one normalized data set, processing and storing transactional data from the extracted practice management data, and analyzing the normalized data set and transactional data, and responsive to an analysis operation, determining a performance measure of the practice being evaluated.

According to one embodiment, the method further comprises one or more practice management data sources relating to the practice being evaluated comprise one or more of a group including financial information, human resource information, patient information, and medical information. According to one embodiment, the act of analyzing the normalized data set and transactional data and responsive to an analysis operation, determining the performance measure of the practice being evaluated includes determining a performance measure related to at least one of a group comprising financial performance, human resource performance, patient performance, and medical performance.

According to one embodiment, the method comprises outputting, by an interface, the performance measure related to at least one of the group comprising financial performance, human resource performance, patient performance, and medical performance to at least one user type or computing entity. According to one embodiment, at least one user type comprises at least one of a group of user types including a practice management user, an outside evaluator user evaluating the practice being evaluated, and an employee user of the practice being evaluated. According to one embodiment, the method comprises an act of interfacing with existing ones or the one or more practice management data sources.

According to one embodiment, the performance measure of the practice being evaluated further comprises at least one of a group including an employee experience index, a client experience index, a financial index, a labor index, an inventory index, a volume index, and a revenue index. According to one embodiment, the performance measure of the practice being evaluated further comprises at least one of a group including root cause, plan information, and a progress toward a target. According to one embodiment, the method comprises a component configured to display the performance measure of the practice, wherein the displayed performance measure may be visualized using at least one of a group including a chart, plot, or table.

According to another aspect, a distributed computer system is provided for managing. The system comprises one or more components configured to extract practice data from one or more practice management data sources relating to a practice being evaluated, extract corporate data from one or more corporate data sources, wherein corporate data includes corporate specifications, integrate the practice data and the corporate data, wherein integration comprises transforming, based on the corporate specifications, the extracted practice management data, determine a performance measure of the practice being evaluated based on the transformed practice management data.

According to one embodiment, the corporate specifications comprise one or more of a group including target data, benchmark data, headquarter data, and reference data. According to one embodiment, one or more practice management data sources relating to the practice being evaluated comprise one or more of a group including financial information, human resource information, patient information, and medical information. According to one embodiment, extracting practice management data comprises mapping the practice management data into at least one normalized data set. According to one embodiment, the mapping of practice management data into at least one normalized data set includes using a general ledger, and the general ledger includes categories having at least one of a group including revenue, cost of goods sold, cost of services, cost of goods service by position, operating expense, gross profit, earnings before interest, taxes, depreciation and amortization (EBITDA), net income, and ownership.

According to one embodiment, the performance measure of the practice being evaluated further comprises at least one of a group including root cause, plan information, and progress toward a target. According to one embodiment, one or more components is further configured to generate a report including the performance measurement.

According to one embodiment, the system comprises a component configured to display the performance measure of the practice. According to one embodiment, the displayed performance measure may be visualized using at least one of a group including a chart, plot, or table.

According to one aspect, a method for managing practice data is provided. The method comprises extracting practice data from one or more practice management data sources relating to a practice being evaluated, extracting corporate data from one or more corporate data sources, wherein corporate data includes corporate specifications, integrating the practice data and the corporate data, wherein integration comprises transforming, based on the corporate specifications, the extracted practice management data, determining a performance measure of the practice being evaluated based on the transformed practice management data.

According to one embodiment, the corporate specifications comprise one or more of a group including target data, headquarter data, and reference data. According to one embodiment, one or more practice management data sources relating to the practice being evaluated comprise one or more of a group including financial information, human resource information, patient information, and medical information. According to one embodiment, extracting practice management data comprises mapping the practice management data into at least one normalized data set.

According to one embodiment, the mapping of practice management data into at least one normalized data set includes using a general ledger, and the general ledger includes categories having at least one of a group including revenue, cost of goods sold, cost of services, cost of goods service by position, operating expense, gross profit, earnings before interest, taxes, depreciation and amortization (EBITDA), net income, and ownership. According to one embodiment, the performance measure of the practice being evaluated further comprises at least one of a group including root cause, plan information, and progress toward a target. According to one embodiment, the method further comprises generating a report including the performance measurement.

According to one embodiment, the method further comprises a component configured to display the performance measure of the practice. According to one embodiment, the displayed performance measure may be visualized using at least one of a group including a chart, plot, or table.

Still other aspects, examples, and advantages of these exemplary aspects and examples, are discussed in detail below. Moreover, it is to be understood that both the foregoing information and the following detailed description are merely illustrative examples of various aspects and examples and are intended to provide an overview or framework for understanding the nature and character of the claimed aspects and examples. Any example disclosed herein may be combined with any other example in any manner consistent with at least one of the objects, aims, and needs disclosed herein, and references to “an example,” “some examples,” “an alternate example,” “various examples,” “one example,” “at least one example,” “this and other examples” or the like are not necessarily mutually exclusive and are intended to indicate that a particular feature, structure, or characteristic described in connection with the example may be included in at least one example. The appearances of such terms herein are not necessarily all referring to the same example.

### BRIEF DESCRIPTION OF THE DRAWINGS

Various aspects of at least one embodiment are discussed herein with reference to the accompanying figures, which are not intended to be drawn to scale. The figures are included to provide illustration and a further understanding of the various aspects and embodiments and are incorporated in and constitute a part of this specification but are not intended as a definition of the limits of the invention. Where technical features in the figures, detailed description or any claim are followed by reference signs, the reference signs have been included for the sole purpose of increasing the intelligibility of the figures, detailed description, and/or claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. In the figures:

FIG. 1 is a representation of a computer system capable of performing various management functions according to various embodiments;

FIG. 2A is a general block diagram of a management system configured to perform one or more management functions relating to a practice having one or more enterprise data sources according to various embodiments;

FIG. 2B shows an example process for managing practice information according to various embodiments;

FIGS. 3A-3B show is an example representation of an architecture according to various embodiments illustrating primary components of the system showing information exchange relationships among a management hub and individual veterinary practice computer systems according to various embodiments;

FIG. 3C shows a high-level data model that may be used according to various embodiments;

FIGS. 4A-4B show a digital transformation process of practice management information according to various embodiments;

FIG. 5 shows an example representation of a dashboard established with the system of the present invention accessible by one or more management users;

FIG. 6 shows another example representation of a dashboard established with the system of the present invention accessible by one or more management users;

FIG. 7 shows an example schema generation using microservices that may be used to implement various aspects;

FIGS. 8A-8C shows an example representation of another computer system capable of performing various management functions according to various embodiments;

FIGS. 9A-9C shows an example representation of a computer system capable of normalizing data from a management system according to various embodiments;

FIGS. 10A-10B shows an example flow diagram of the report distribution according to various embodiments;

FIGS. 11A-11B shows an example representation of a computer system capable of report distribution and viewing according to various embodiments;

FIGS. 12A-12B shows an example representation of an architecture according to various embodiments for revenue integration; and

FIG. 13A shows an example interface configured to display a daily flash report for the practice according to various embodiments;

FIG. 13B shows another example interface configured to display a daily flash report for the practice according to various embodiments;

FIGS. 14A-1 and 14-2 shows an example interface configured to display a weekly flash report for the practice according to various embodiments;

FIG. 14B shows an example interface is configured to display visual representations of metrics according to various embodiments;

FIG. 14C shows another example interface configured to display visual representations of metrics according to various embodiments.

FIG. 14D shows a third example interface configured to display visual representations of metrics according to various embodiments.

FIGS. 15A-15B shows an example interface configured to display a monthly flash report for the practice according to various embodiments;

FIGS. 16A-16B shows an example interface configured to display a category and patient flash report for the practice according to various embodiments;

FIGS. 17A-17C shows an example interface configured to display a weekly management flash report for various practice according to various embodiments;

FIGS. 18A-18B shows an example interface configured to display a table including metrics associated with individual practices according to various embodiments;

FIGS. 19A-19B shows an example interface configured to display a dashboard relating to a metric according to various embodiments;

FIG. 20 shows an example interface configured to display a metric over time according to various embodiments; and

FIGS. 21A-21B shows an example process for acquisition of a new practice according to various embodiments.

### DETAILED DESCRIPTION

According to various embodiments, systems and a platform are provided that enables end-users to gain insight into group and individual practices. Those insights may be obtained for any desirable aspect of each practice and the collection of practices including, but not limited to, financial information, medical information, human resources information, and patient information. That information can be used by group management to identify best practices, and it can be used to advise individual practices on strengths to be reinforced and weaknesses to be addressed. That information may be conveyed to group management and individual practices in the form of dashboards customizable to the particular functions of specific users and their personas.

### Overview

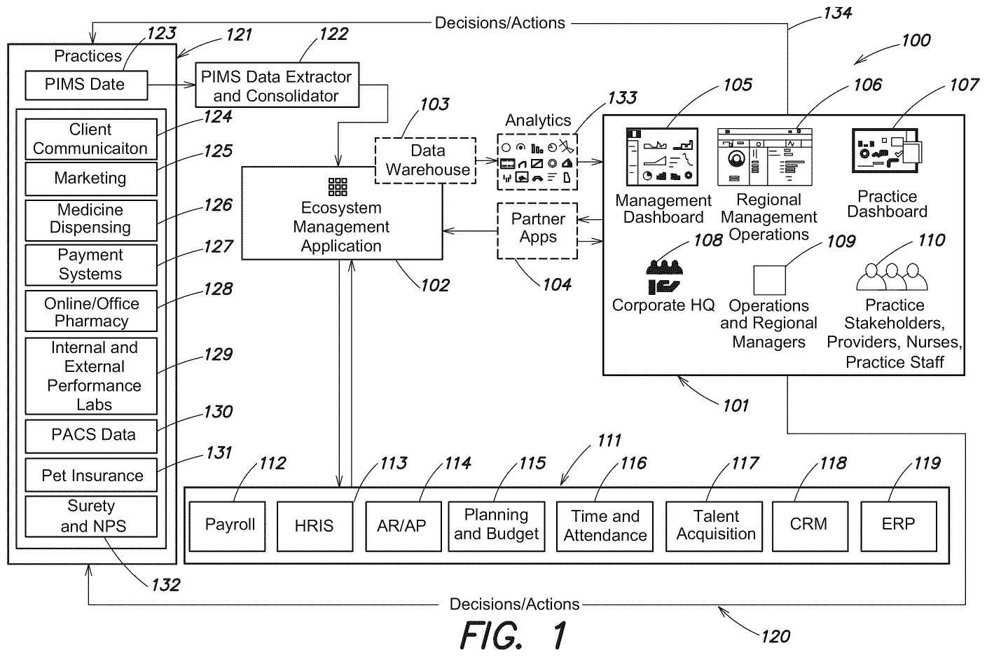

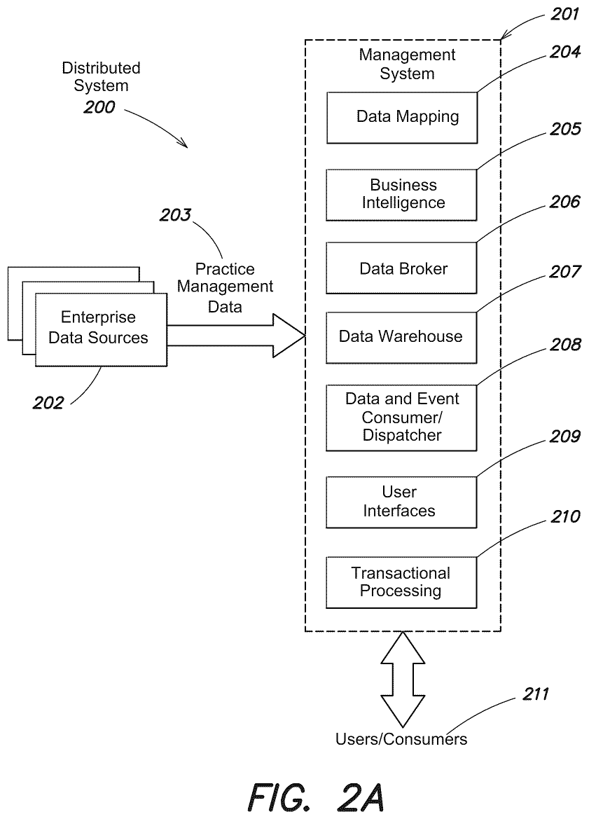

In some embodiments, an ecosystem is provided that includes an integrated software platform that enables data exchange, integration, automation and analytics. FIG. 1 is a representation of a distributed computer system capable of performing various management functions according to various embodiments.

In some implementations, the ecosystem 100 is implemented across disparate SAAS software fulfilling practice and management business workflows (e.g., of an aggregator of practices). A core management application (e.g., Ecosystem Management Application 102) along with mesh of proprietary microservices sit at the core of this ecosystem 100. The system elements house reference data, translation logic and configuration which enables the platform to be highly automated and interoperable.

In some embodiments, the ecosystem 100 comprises a number of subsystems including practice software 121, corporate software 111, ecosystem management application 102 and associated data and services, and user-facing analytics 101 and partner applications 104

### Practice Software

Practice software 121 may include, for example, software applications (e.g., installed on premise or SAAS-based) which are used at the practice to carry out daily operations for the practice. For instance, a Practice Management System (PMS) or Practice Information Management System (PIMS) is the most critical software at any veterinary practice. The PMS is either installed as a Client/Server application locally or SAAS based. In some embodiments as described herein, existing PMS systems are integrated as part of an ecosystem (e.g., ecosystem 100) for the purpose of more effectively managing and evaluating the practice.

To this end, data from the PMS is extracted and normalized (e.g., via a 3^{rd} party (PIMS) data extractor and consolidator 122) at a regular interval (e.g., daily) and staged in the cloud to be consumed by ecosystem applications. Besides PMS, other software used at practice on a regular cadence to fulfil specific workflows may include Picture Archiving and Communication Systems data (PACS data) (e.g., element 130), Wellness, Client Communication (e.g., element 124), Marketing (e.g., element 125), Medicine Dispensing (e.g., element 126), Payment Processing (e.g., element 127), Pharmacy (e.g., element 128), Insurance (e.g., element 131), Labs (e.g., element 129), Surety (e.g., element 132), among others. Data from each of these software are loaded at a regular cadence into a respective online transaction database and may be piped to a data warehouse (e.g., element 103) via any communication method (e.g., file exchange, rest services, or other transfer method).

### Corporate Software

Further, the ecosystem may include one or more integrated applications configured to work with one or more corporate applications. For instance, one or more SAAS-based software applications may be provided that work independently or in conjunction with one or more software entities at a corporate headquarters (e.g., the aggregator of practices) to fulfill business processes for a corporate entity. For example, the system may comprise SAAS-based solutions 111 that are used to generate data in their respective domains which is consumed by an ecosystem management application (e.g., system 102) at a regular cadence or on demand. Corporate-level SAAS solutions 111 may include, for example, Human Resources Information System (HRIS) (e.g., element 113), Payroll (e.g., element 112), Accounts Receivable/Accounts Payable (AR/AP) (e.g., element 114), Budget/Planning (e.g., element 115), Time/Attendance (e.g., element 116), Scheduling, Talent Acquisition (e.g., element 117), Customer Relationship Management (CRM) (e.g., element 118), Enterprise Resource Planning (ERP) (e.g., element 119), among other corporate-level applications.

### Ecosystem Management Application

In some embodiments, an ecosystem management application (e.g., element 102) is provided that integrates corporate software 111 with practice software 121 to enable automation, analytics and hybrid efficient business workflows reduces time and improving overall service quality. In some embodiments, the ecosystem management application 102 is implemented as proprietary microservices hosted in cloud that integrate these disparate software systems.

In some embodiments, these microservices abstract integration between software system by centrally housing translation logic and reference data as configuration, thereby making resultant platform a plug and play software platform. According to some embodiments, any practice 121 or corporate level SAAS application 111 can be replaced without impact to overall user experience or underlying business automation by changing the configuration only. The system, in some implementations, includes a data warehouse (e.g., data warehouse 103). In some embodiments, data persisted (e.g., in microservice-based polyglot online transaction processing (OLTPs)) are ultimately piped and persisted in the data warehouse. In some implementations, some data transformations are applied to the piped data. The microservices and data warehouse for the platform provides a solid backend system for customer facing applications (e.g., element 101).

### Customer-Facing Analytics and Partner Applications

Data from the data warehouse (e.g., element 103) and services are fed into an analytics engine (e.g., analytics engine 133) to generate meaningful information that may be used for reports, charts, decisions and actions. In some embodiments, charting and reporting may be fulfilled by an interface component 101 (e.g., a 3^{rd} party business intelligence tool) which plugs into ecosystem management application 102. The ecosystem management application 102 may also serve as a platform to build highly effective customer facing applications (e.g., partner apps 104) and provide hybrid consumable webservices for fulfilling complex business processes, Analytics, reports, charts and applications may be customized to be consumed by stakeholders at various roles and levels.

The system may include a number of interfaces and applications to be consumed by various user types and computing entities (e.g., other computer systems). For instance, the system may provide one or more interfaces for managing the various practices being managed within the ecosystem. For instance, the system may include a management dashboard 105 that permits managing users to manage the system. The system may also include a regional management operations dashboard 106 that reports on multiple practices in an area/region. The system may also include a practice dashboard 107 that permits managing users to make decisions and view operational performance regarding a specific practice. The system may include other interfaces, such as a corporate HQ interface 108, an operations and regional managers interface 109, and practice stakeholders interface 110 that permit various users to view performance of the practice at various levels and perspectives.

In some embodiments, based on the information presented by the customer facing application 101, managing user may make decision/actions 120, 134 regarding one or more practices. In some implementations, the system may automatically implement a decision or action regarding one or more practices based on information abstracted from previous engagements with other practices. In some embodiments, the ecosystem 100 may be configured to continue to collect data after a decision/action 120, 134 has been implemented. The ecosystem 100 may analyze the data to further inform managing users about the impact and effects of the decision/action 120, 134.

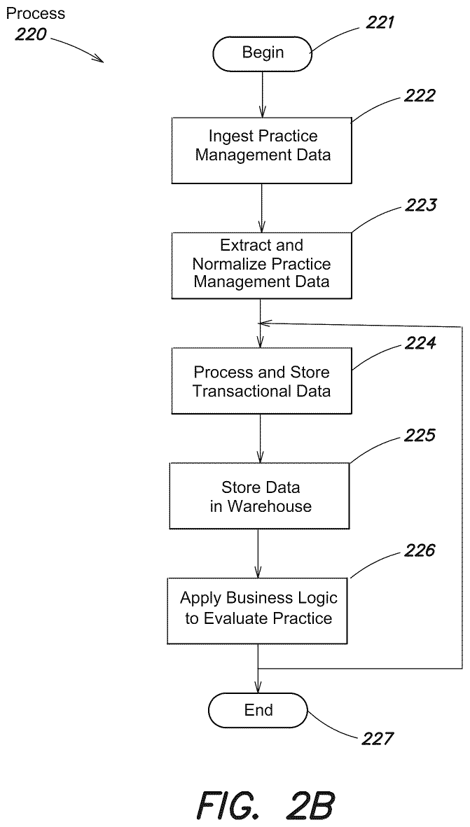

FIG. 2A is a general block diagram of a management system configured to perform one or more management functions relating to a practice having one or more enterprise data sources (e.g., PIMS, client communication, payment system, etc.) according to various embodiments. In particular, FIG. 2A shows a distributed computer system 200 that shows an overall architecture of a management system capable of managing one or more practices. For example, the system may include a management system 201 that is capable of interfacing with one or more practice computer systems (e.g., more generally, enterprise data sources 202). Management system 201 may receive from one or more enterprise data sources practice data 203, used for the purpose of managing, and providing additional functionality for analyzing one or more practices (e.g., a veterinary practice). For example, while not shown, management system 201 may include the ecosystem management application 102 as described above with reference to FIG. 1.

Management system 201 may include a number of computer-based components that perform one or more functions within the distributed system 200. For instance, as discussed above with reference to FIG. 1, the system may include one or more services that perform various functions within the distributed system. For example, system 201 may include a data mapping component 204 that provides data mapping functions from the received practice management data 203 information that may be consumed and processed by the system 201. In some embodiments, the data mapping component 204 may map extracted practice management data into an analytics engine to be analyzed. In some embodiments, the data mapping component 204 may map extracted practice management data into at least one normalized data set to be analyzed. The data mapping component may utilize translation logic and reference data to map the extracted practice management data.

Further, system 201 may include a business intelligence component 205 that interprets one or more data elements relating to the practice management data 203. In some embodiments, the business intelligence component 205 may produce analytics and reports that may be consumed by users. In some embodiments, the business intelligence component 205 may produce metrics and indices for understanding various aspects of at least one practice. The system may also include a data broker component 206 that manages the transfer of data between the conventional practice management systems (e.g., system 121) and the ecosystem management application 102. Data broker component 205 may also manage the transfer of data between corporate applications (e.g., system 111) and the ecosystem management application 102. Practice management data 203 or corporate application data may be stored in various formats within a data warehouse 207. Further, system 201 may include a component (e.g., component 208) that consumes and dispatches data and events from/to one or more systems. In some embodiments, the component 208 may dispatch data and events from/to revenue services, marketing services, online pharmacy services, partner applications, data transformation services, etc.

As discussed above, the system may include one or more user interfaces (e.g., interfaces 209) that provide information relating to the management of the practice to one or more user types. In some embodiments, one or more of the user interfaces 209 may permit managing users to make decisions regarding at least one practice. Further, the system may include one or more transactional processing components (e.g., element 210) that is used to receive and process transactions that occur within the practice. As discussed, the system may be used to manage practices in a real time manner using real-time transactional data.

FIG. 2B shows an example process for managing practice information according to various embodiments. For instance, systems (e.g., systems 102, 201) may be used to perform one or more operations using practice management data received from one or more practice-based systems. At block 221, process 220 begins.

At block 222, the system ingests practice management data. For instance, this may be accomplished using one or more interfaces and one or more computer-based entities that are adapted to receive practice management data from one or more enterprise sources. According to some embodiments, practice management data may be ingested automatically on a scheduled cadence (i.e., daily) or on demand. At block 223, the system extracts and normalizes the practice management data. For instance, the information may be reformatted and normalized to be used within an ecosystem management application. Further, at block 224, the system may be configured to process and store transactional data associated with one or more transactions performed within the practice. Received information may be stored within a data warehouse at block 225. The system may be used to apply business logic to the received data to evaluate the practice at block 226. Optionally, information determined by the business logic may be presented to one or more users within a user interface for the purpose of managing the practice. At block 227, process 220 ends. However, it should be appreciated that the system may be operated continually to manage practice performance information over periods of time. For instance, a change in practice management may be implemented in response to the evaluation of the practice at block 226. Data may be collected after this change was implemented to evaluate the practice and how the change impacted the practice management. As such, this may provide a feedback loop for managing the practice.

### High Level Architecture

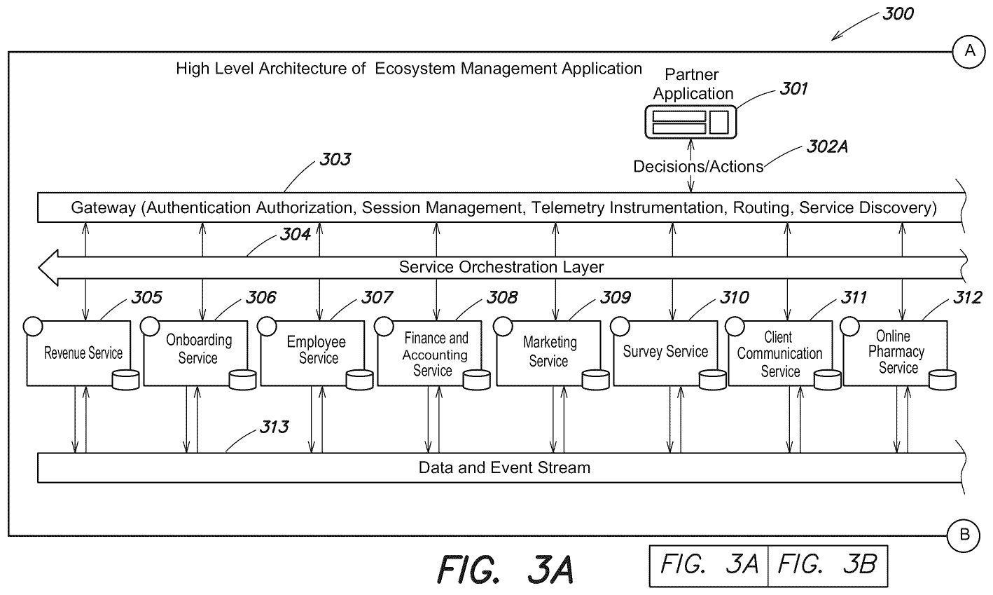

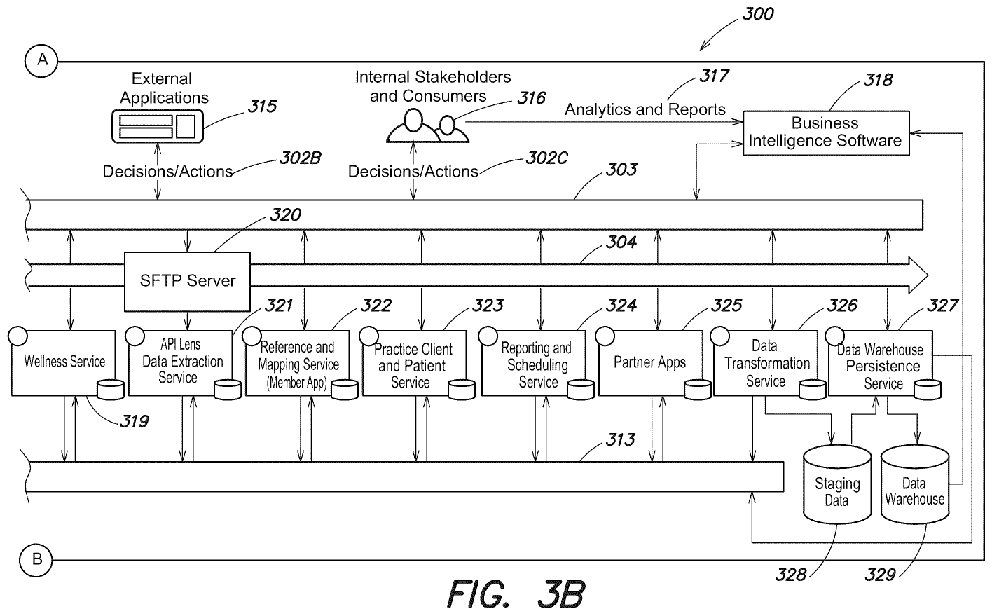

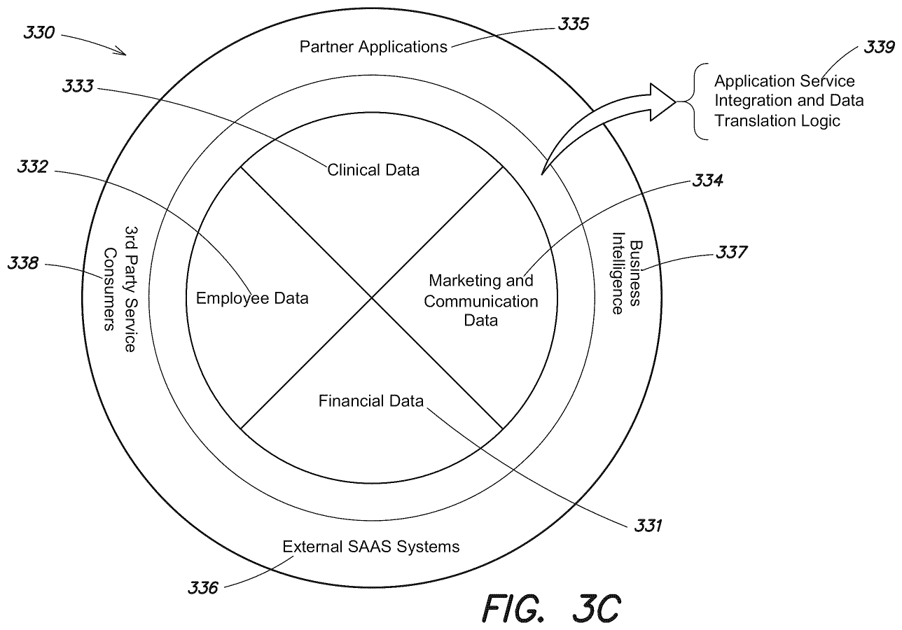

FIGS. 3A-3B show is an example representation of an architecture according to various embodiments. In particular, FIGS. 3A-3B show primary components of the system (e.g., an ecosystem management application 300) showing information exchange relationships among a management hub and individual veterinary practice computer systems according to various embodiments.

This diagram represents consumption, orchestration and data exchange layers of microservices of the ecosystem management application 300. In some embodiments, this is a microservices-based architecture including certain levels of synchronized and asynchronized data exchanges. In some implementations, each service exists independently with its own backend transactional data base. Multiple services have the capability to be orchestrated in conjunction via a service orchestration layer 304 and can also exchange data via data and event stream 313. In some instances, data generated at individual transactional databases gets piped into the data warehouse (e.g., element 329) directly or via staging data (e.g., element 328) with or without transformation, as needed. The gateway layer 303 serves external applications and is used for cross-cutting concerns like logging, session management, security, load balancing, etc.

As shown, system 300 may interface with one or more users and systems. For instance, system 300 may interface with one or more partner applications 301 that receive/send one or more decisions/actions 303A. Further, system 300 may interface with one or more external applications 315 that receive/send one or more decision/actions 302B. Further, system 300 may interface with one or more internal stakeholders and consumers 316 (e.g., users) and may be configured to receive/send one or more decision/actions 302C.

System 300 may include one or more components that perform analytics and determine various outputs for users and systems. For example, system 300 may include a business intelligence software component 318 that produces analytics and reports 317 that may be consumed by users and systems.

As discussed, system 300 may include one or more services that are used to manage one or more aspects of the practice being managed. For example, the system may include a wellness service component 319, an application programming interface (API) lens data extraction service 321, a reference and mapping service 322, a practice client and patient service 323, a reporting and scheduling service 324, one or more partner applications 325, a data transformation service 326 and a data warehouse persistence service 327. Further, system 300 may include a revenue service component 305, and onboarding service component 306, an employee's service component 307, a finance and accounting service component 308, a marketing service 309, a survey service component 310, a client communication service component 311, and a pharmacy service component 312.

### High-Level Data Model

FIG. 3C shows a high-level data model 330 that may be used according to various embodiments. In some embodiments, the high-level data model 300 comprises broad categories of data—clinical data 333, employee data 332, marketing and communication data 334 and financial data 331. In some implementations, the data is persisted throughout a transactional database and data warehouse in different states for efficient retrieval and meaningful information to represent labor and financial indices, employee engagement key performance indicators (KPIs), communication metrics, marketing analytics, among other information used to manage the practice and observe practice performance.

In some embodiments, the clinical data model 333 supports electronic health records, administrative data, diagnosis, wellness, among other clinically-related information. The employee data model 332, in some embodiments, supports HRIS, payroll and earning records, performance, licenses, learning, etc. In some implementations, the financial data model 331 supports revenue, cost of service and goods, inventory data, among other financial information relating to the practice. The marketing and communication data model 334 may support communication volume, Net Promoter Score (NPS), web marketing, and other information relating to marketing and communication activities.

The translation logic data model may, in some implementations, contain an application service integration and data translation logic 339. The application integration and data translation logic 339 may include application integration definitions (e.g., for 3^{rd} party SAAS applications 336), reference data to be able to translate incoming SAAS information (e.g., for 3^{rd} party SAAS applications) into consumable versions for the ecosystem management application and outgoing information from ecosystem microservices to consumable SAAS versions (e.g., for 3^{rd} party consumers 338). In some embodiments, business intelligence application 337 is configured to read data directly from warehouse with minimal transformation or references if needed. The data model may also be configured to generate data for partner applications 335.

### Metrics to Process Improvement

In some embodiments, it is appreciated that metrics that are standardized across practices may be used to improve processes within any one practice. A system that automatically identifies performance gaps may be used to identify issues and improve process performance within the practice.

### Metrics

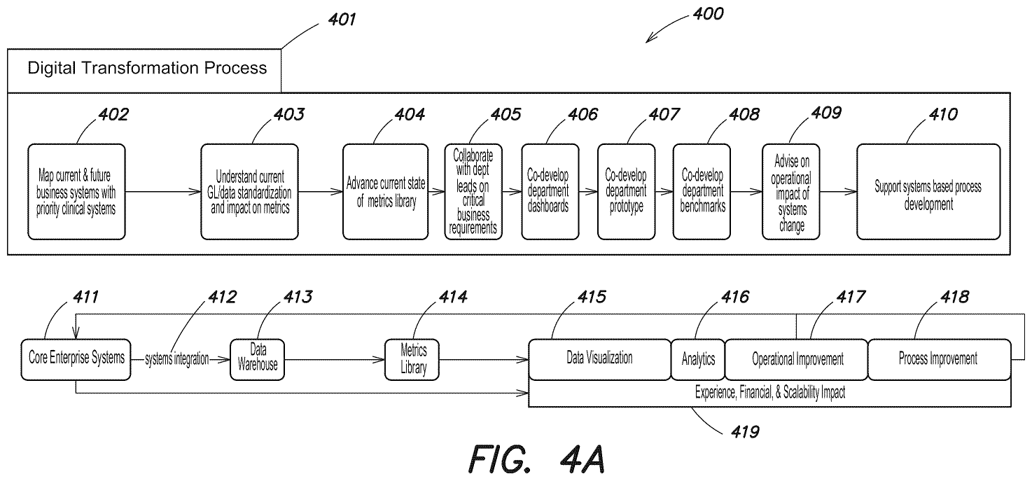

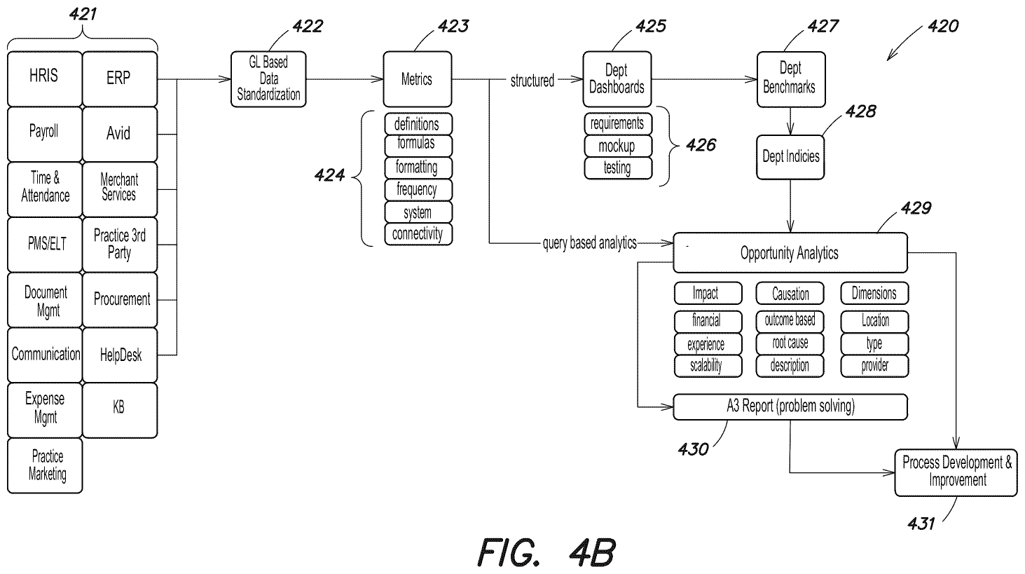

FIGS. 4A-4B show a digital transformation process of practice management information according to various embodiments. Translating the four data categories described above with reference to FIG. 3C into actionable insight and process improvement requires a deep understanding of what drives business and clinical performance. According to some embodiments, a metrics library may be used that is derived from the four data categories. Each metric is defined by six attributes: definition, formula, format, frequency, system, and connectivity. Based on department requirements and focus the metrics are visualized in structured drillable dashboards and tied to department benchmarks.

### Indices

For multiparameter analysis, associated metrics may be grouped into department indices. Based on importance and impact, metrics may be weighted, and algorithms are provided that define how metrics perform to benchmarks. In some embodiments, these indices provide rapid visualization of associated metrics with predefined algorithms for consistent benchmarking across business and clinical teams.

### Opportunity Analytics

Understanding the impact, causation and dimensions of a performance gap are critical to predicting client and employee behavior and the implementation of operational and process improvements. Querying the database (e.g., a data lake) may provide impact and dimension analysis, but causation requires input from frontline teams in the form of root cause. It also allows the managing users to suggest and implement a corrective plan. In some embodiments, such inputs are provided and presented to managing users as outputs (e.g., within a management interface such as those discussed below).

FIG. 4A shows a process 400 for implementing various aspects of the management system including a digital transformation process 401. In particular, process 400 may include one or more steps including those that capture expert heuristics, information relative to business requirements and specific practices, and capturing those within the ecosystem. In particular, a digital transformation process 401 includes an act of mapping current and future business systems with priority clinical systems at block 402, determining and understanding current GL (General Ledger) data standardization and impact on particular metrics at block 403, and at block 404, advancing the current state of the metrics library.

Further, experts may collaborate with department leads on critical business requirements at block 405, develop department dashboards associated with department leads at block 406, codeveloped department prototypes at block 407, codeveloped department benchmarks at block 408, advise on operational impact of systems change 409 and at block 410, support systems-based process development.

Regarding data transformation, neither originates at block 411 from core enterprise systems associated with the practice manage systems integration function 412 operates to transfer and adapt information from the core enterprise systems 411 into data that can be stored within data warehouse 413. At block 414, metrics associated with the metrics library are applied, and the system may perform a number of operations that aid a managing user in improving the practice and its performance. For example, the system provides experience, financial, and stability impact 419 through data visualization 415, presentation of analytics 416, recommendations on operational improvements 417, which result in observed process improvements 418. Using the experience, financial and stability impact 419, managing users may implement decisions/actions. After a decision/action may be implemented, data may be continued to be collected and analyzed to inform managing users about the impact and effects of the decisions/actions.

FIG. 4B shows a more detailed application of metrics, interfaces, and may be used within the system to improve the performance of practice will organizational components. Information provided by enterprise systems 421 are provided to GL-based data standardization 422 process. The standardized data processed by one or more metrics 423 including a number of attributes 424 including definitions, formulas, formatting, frequency, system and connectivity. Based on department requirements and focus, the metrics are visualized within department dashboards 425 and tied to department benchmarks 427. Associated metrics are grouped into department indices 428.

Query-based analytics and department indices are fed into opportunity analytics 429, and reporting (e.g., A3 reporting (problem solving)) and output analytics may be generated that permit process development and improvement at block 431.

### Data Visualization, Root Cause and Plan

As discussed, the system may provide information to users at all different levels in order to provide practice improvements. Improvement at scale in a complex multiunit service environment requires informed, engaged and empowered employees. According to some embodiments, a practice performance dashboard interface may be provided that uses metrics (e.g., hundreds of metrics in various categories), the metrics when used in isolation provide a very limited understanding of the business and people, but when indexed, curated and visualized with root cause and plan then meaningful and targeted change can be implemented.

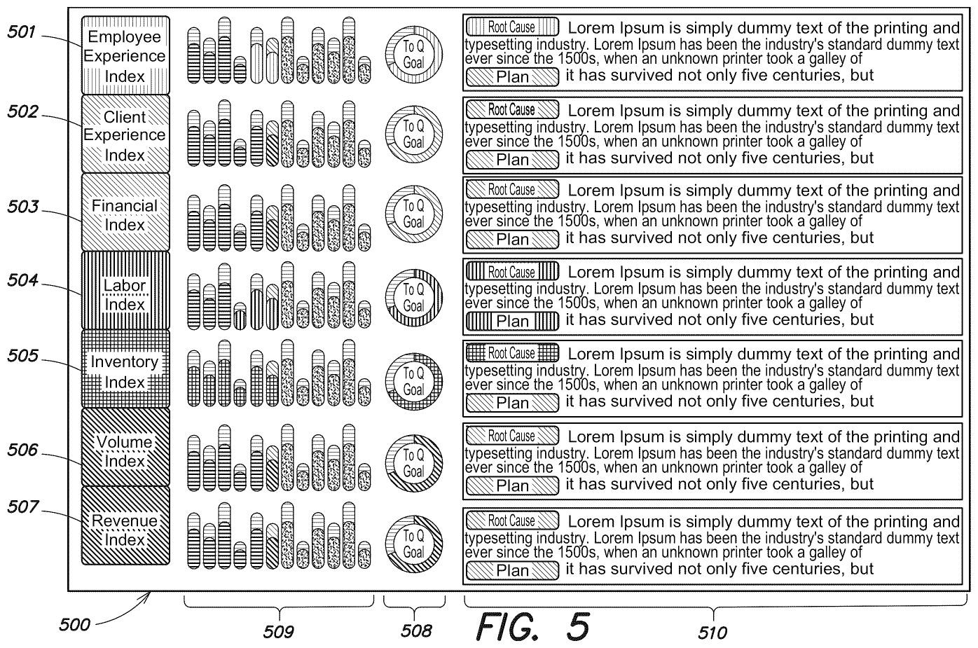

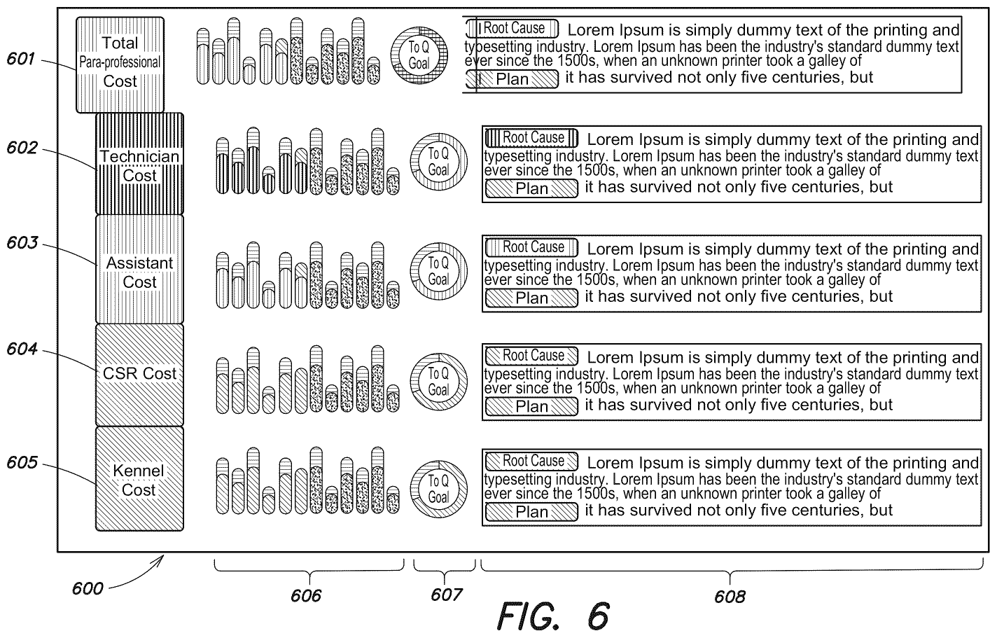

FIGS. 5 and 6 shows an example representation of a dashboard established with the system of the present invention accessible by one or more management users. In Particular, FIG. 5 includes an interface 500 that is configured to display a number of indices related to performance of the practice. Further, interface 500 may include a number of other indices that show in particular indexed to a particular goal, along with causes of the performance, and plan indications that may to process improvement.

As discussed, a number of aspects relating to metrics may be indexed, curated, individualized with root cause and plan information. For example, metrics associated with an employee experience may be indexed and displayed as employee experience index information 501 within interface 500. Metrics associated with an employee experience index may include number of surgeries performed. Further, interface 500 may include a number of other indices such as client experience index 502, financial index 503, labor index 504, inventory index 505, volume index 506, revenue index 507, and price index. In some embodiments, metrics associated with the volume index 506 may include invoice counts by hospital, invoice counts by time, and patient visit count. Metrics associated with the revenue index 507 may include total revenue by hospital, total revenue by time, and percent change over periods. Metrics associated with the price index may include average client transaction.

Interface 500 may also include a display area 509 indicating various metrics that were measured in relation to a rolled-up index. The interface, in some embodiments, allows the user to quickly identify aspects of the practice that relate to a metric for a particular index. In some cases, the performance of any metric for a particular index may be indicated by one or more colors, patterns, or other indicator types. Interface 500 may also include an indicator 508 that provides progress information towards a particular goal (e.g., one that is agreed to by the practice). The system may also include a portion of the interface 510 that displays root cause information for particular indices for performance measures along with suggested plan information. Such cause information and plan information may be abstracted from previous engagements with other practices and may be applied automatically to the system being evaluated.

FIG. 6 shows another example interface implementation associated with the labor index interface (e.g., labor index 504 of FIG. 5). According to some embodiments, a user is permitted to select an interface element (e.g., the labor index element 504) and migrate down to lower-level interfaces to see specifics at various levels. For instance, a selection of the labor index element 504 may lead to a practice labor dashboard that breaks out practice labor into a number of different categories, one of which may be total paraprofessional cost. As shown in FIG. 6, upon selection of a total paraprofessional cost interface element from a previous screen, and interface 600 may be presented to the user that breaks out various paraprofessional cost information associated with different roles. Therefore, analysis provided by the system may be hierarchical in that metrics data is provided in varying levels within various dashboards.

The FIG. 6 interface 600 may itself include one or more interface elements including total paraprofessional cost elements 601, which breaks down to technician cost index element 602, assistant cost index element 603, client service representative (CSR) cost index element 604, and kennel cost index element 605. Each of the indexes may have associated metrics elements 606, goal elements 607, and root cause and plan information 608. In this manner, users can drill down into various issues and quickly isolate particular performance issues in an efficient manner.

### Data Transformation and Schema Creation

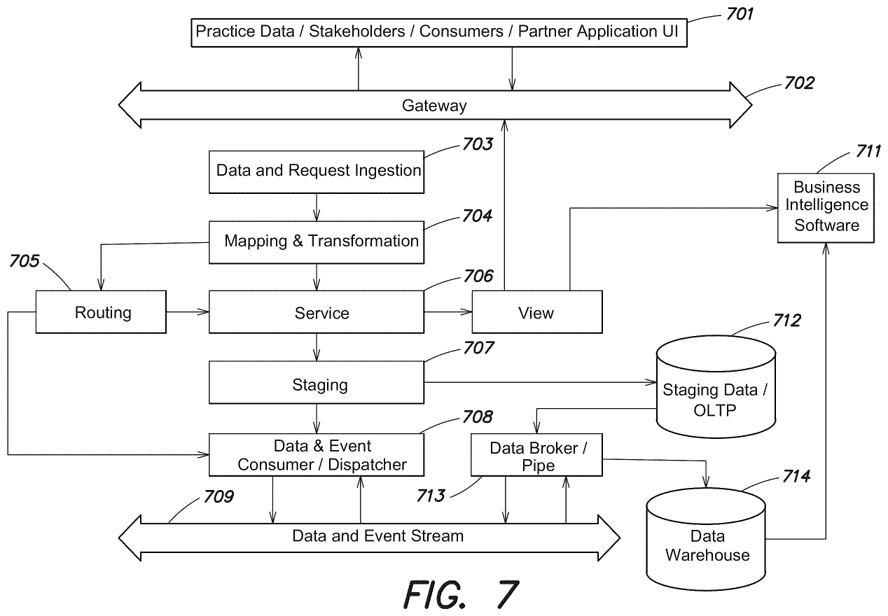

FIG. 7 shows an example process for creating a schema used to implement various aspects. In an initial connection to a legacy practice, data connections are made to existing enterprise systems where data is received and there is a request for data ingestion a block 703. Received data is mapped and transformed at block 704 where it is routed at block 705 and various services 706. The data may be staged at block 707 and stored within an online transactional database at block 712. Data and information related to events may be consumed or dispatched at block 708 and placed on a data stream 709. Data and event information from the data and events stream may be processed by one or more data brokers/pipes which are then stored in data warehouse 714. Further, transactional data stored within database 712 may also be processed and stored by one or more data brokers/pipes 713 within data warehouse 714.

Data stored within data warehouse 714 may be used, for example, by business intelligence software 711 to provide information regarding the practice to one or more users (e.g. stakeholders, data consumers, or other user types) within various user interfaces 701. In some embodiments, various services 706 may provide information regarding the practice to one or more users within various user interfaces 701. Such information may be provided through a gateway 702 or more information consumers.

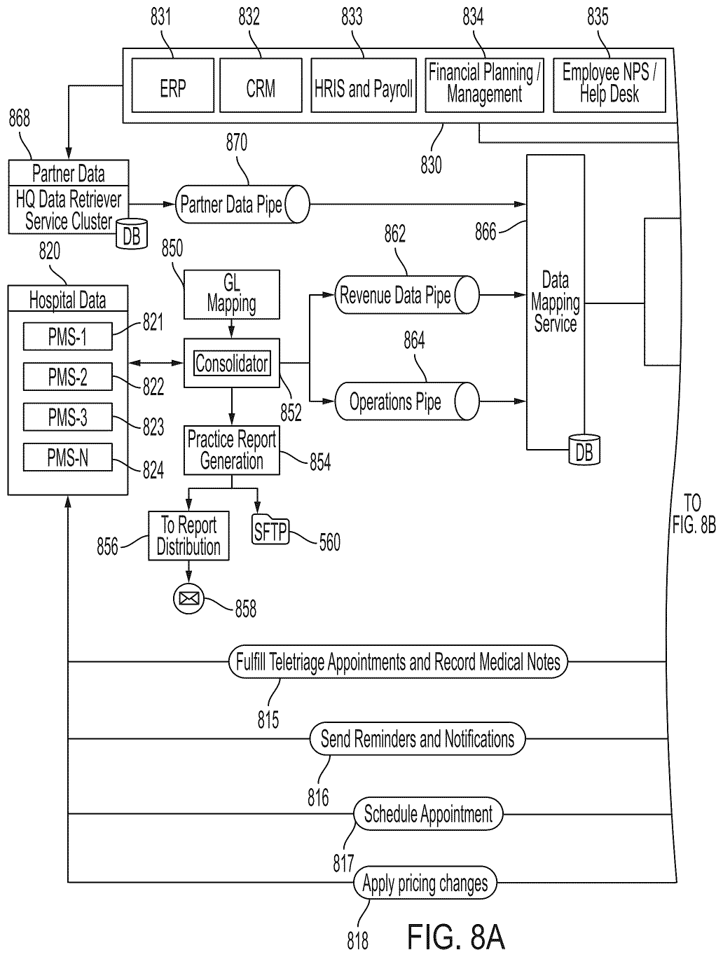

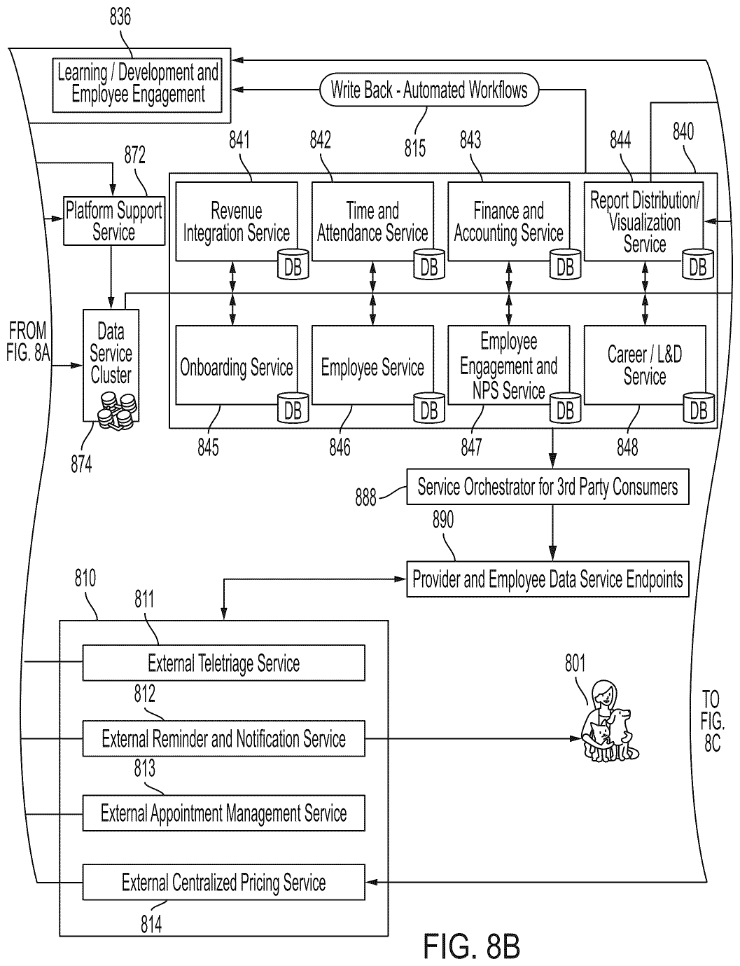

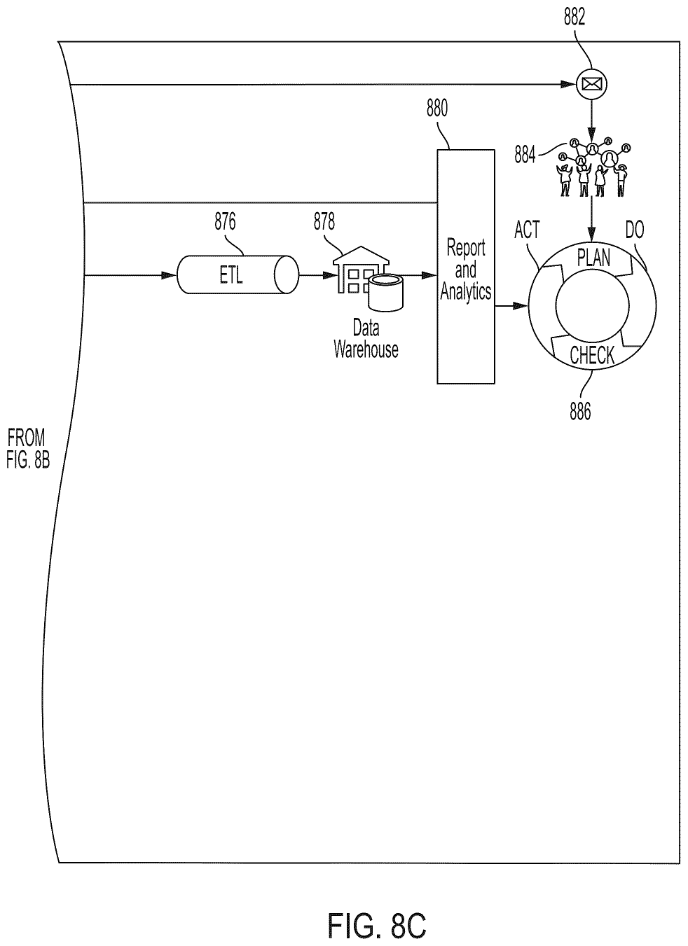

FIGS. 8A-8C is a representation of another distributed computer system capable of performing various management functions according to various embodiments. In some embodiments, the system 800 may aggregate practice information and corporate information from disparate SAAS software to enable data exchange, integration, automation, and analytics.

System 800 may include practice software configured to collect and/or receive practice data (e.g., hospital data 820). The practice software may include at least one PMS 821-824 for effectively managing and evaluating the practice. Other practice software may be used within the system 800 and is configured to receive data from external services 810. For instance, external services may include external teletriage services (e.g., element 811), external reminder and client notification services (e.g., element 812), external appointment management service (e.g., element 813), external centralized pricing service (e.g., element 814), among other external services. The external services 810 may provide information to the practice, such as full teletriage appointments and record medical notes (e.g., element 815), sent reminders and notifications (e.g., element 816), scheduled appointments (e.g., element 817), pricing changes (e.g., element 818), among other services. Other practice software may be used to fulfil specific workflow, which may include PACS data, wellness, client communication, etc.

Data collected and/or received from the practice software may be extracted and normalized at a regular interval (e.g., daily) or on demand. In some embodiments, practice data may be extracted from the practice software and normalized by consolidator 852. The consolidator 852 may be further configured to receive GL mapping 850 and apply GL mapping 850 to the extracted data for normalization of the practice data. In further implementations, the practice report generation 854 may apply business intelligence tools to the extracted and normalized data for creating a practice report. The practice report may be distributed via an email service 856, 858 or a secure file transfer protocol (SFTP) 860.

System 800 may also include corporate software 830. The corporate software 830, for instance, may be one or more SAAS-based software applications that may work independently or in conjunction with one or more software entities at a corporate headquarters to fulfill business processes for a corporate entity. In some implementations, the corporate software 830 may include ERP (e.g., element 831), CRM (e.g., element 832), HRIS and payroll (e.g., element 833), financial planning and management (e.g., element 834), employee NPS and help desk (e.g., element 835), learning and development and employee engagement (e.g., element 836), among other corporate software.

Further, system 800 may also include multiple services that integrate practice software and corporate software data to enable automation, analytics, and hybrid efficient workflows to reduce time and improve the overall service quality. In some embodiments, the services abstract integration between software systems by centrally housing translation logic and reference data as configuration, thereby making the platform a plug and play software platform. In some embodiments, the services may be included in a management system (e.g., ecosystem management application 102). The services may include a data mapping service 866, platform support service 872, data service cluster 874, and management services 840.

The data mapping service 866 may be configured to receive and map both practice software data and corporate software data. Extracted, normalized, and consolidated practice data may be transformed via a revenue data pipe 862 and/or operations pipe 864. The transformed data may then be piped to a data mapping service 866. Data mapping service 866 may map extracted and normalized practice data to the data service cluster 874 either directly or via the platform support service 872. Data extracted from the corporate software 830 may also be piped to the data mapping service 866. In some embodiments, corporate data may be extracted, normalized, and/or mapped by the platform support service 872 and then piped to the data service cluster 874. In further embodiments, extracted corporate data may be received by the HQ data retriever service cluster 868 and piped to the data mapping service via the partner data pipe 670. The data mapping service 866 may map the extracted corporate data to the data service cluster 874 either directly or via the platform support service 872.

The data from the data service cluster 872 may then be piped to the management services 840 which may be used to manage one or more aspects of the practice being managed. For example, the management services may include revenue integration service (e.g., element 841), time and attendance service (e.g., element 842), finance and accounting service (e.g., element 843), report distribution and visualization service (e.g., element 844), onboarding service (e.g., element 845), employee service (e.g., element 846), employee engagement and NPS service (e.g., element 847), career and learning and development service (e.g., element 848), among other microservices. In some embodiments, data from the management services 840 may be piped into the data warehouse 878. In further embodiments, the management services 840 may provide a write back to the corporate software for automated workflow 892. The management services 840 may also be configured to provide provider and employee data service endpoints 890 via a service orchestrator 888.

Data from the data service cluster 874 is then piped to the data warehouse 878 via an extract, transform, and load (ETL) process 876. In some embodiments, management services 840 may provide data to the data warehouse 878 via the ETL process 876. Data from the warehouse 878 is then fed into a report and analytics engine 880 to generate meaningful information that may be used for reports, charts, decisions and actions. In some embodiments, the charting and reporting may be performed using the report distribution and visualization service 844. In some embodiments, the report distribution and visualization service 844 may send business reports to management users 884 or may provide an interface for the management users 884 to visualize or review the business reports. Based on the report and analytics, managing user 884 may make decisions or actions 886 regarding the one or more practices. In some implementations, the system may automatically implement a decision or action regarding one or more practices based on information abstracted from previous engagements with other practices. In some embodiments, the system 800 may analyze data following implementation of the decision or action to further inform managing users about the impact and effects of the decision or action.

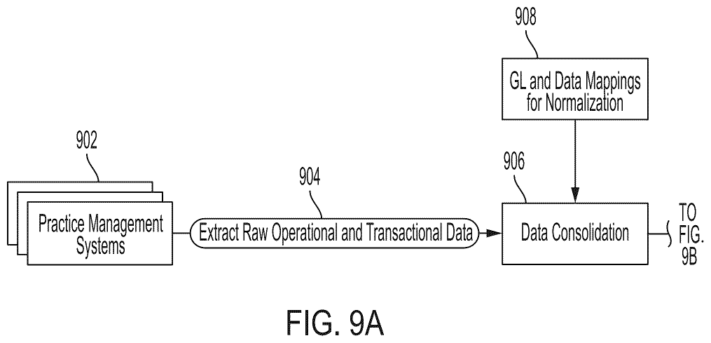

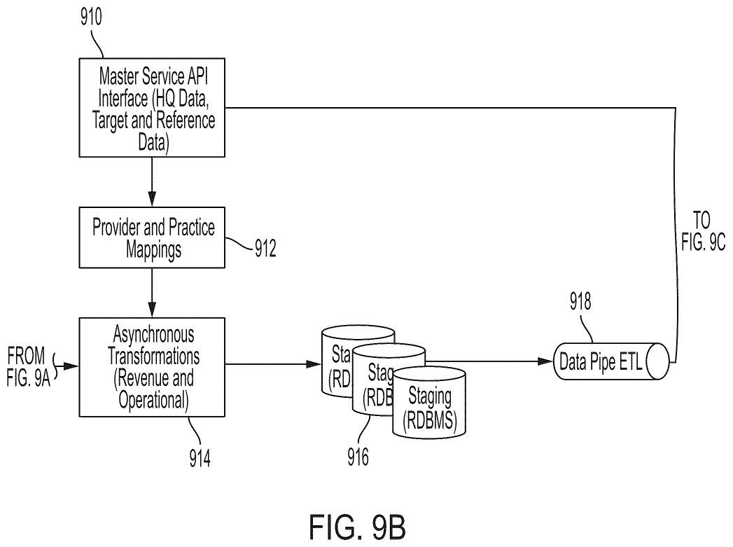

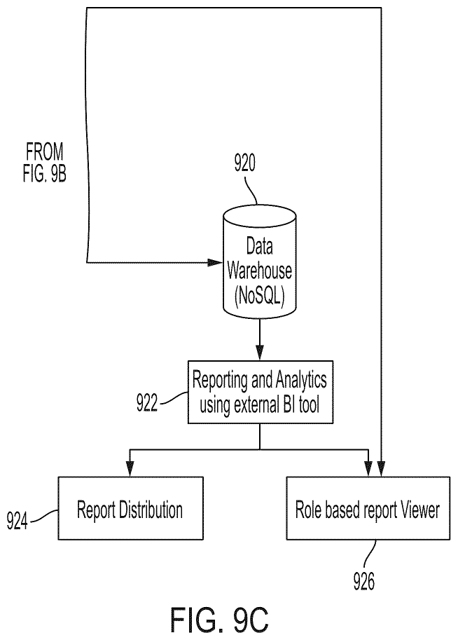

FIGS. 9A-9C is a representation of a computer system capable of normalizing data from a management system according to various embodiments. According to some embodiments, the system 900 may aggregate practice information and corporate information from disparate SAAS software.

System 900 may include a PMS 902 configured to carry out daily operations for the practice. Data (e.g., raw operational data and/or transactional data 904) from the PMS may be extracted on a regular cadence (e.g., daily) or on demand. In some implementations, data consolidation 906 may occur by applying a GL and data mapping 908 the extracted data such that the extracted data is normalized. The normalized data may then be transformed via a revenue and/or operational pipe (e.g., elements 862 and 864). The transformation may be based on provider and practice mappings 912 derived from the master service API 910. The master service API 910 may be configured to interact with managing users and may provide headquarter specifications for transforming the normalized data. The headquarter specifications may include headquarter data, target data, and reference data. The transformation of the normalized data may provide standardization of provider IDs, aggregations, and canned rollups by entities (e.g., department, positions, job titles, hospital, and employee type).

The transformed data may then be staged 916. In some embodiments, the data is staged in a relational database management system (RDBMS). Stage data may then be piped (e.g., using a ETL process 918) into the data warehouse 920. In some embodiments, the data warehouse 920 may be a NoSQL data warehouse. The data from the data warehouse 920 may be fed into an analytics engine to provide reporting and analytics 922. The analytics engine may include external business intelligence tools. The reporting and analytics may then be distributed 924 to managing users via an electronic message or secure file transfer protocol (SFTP). The reporting an analytics may also be displayed 926 to the managing users on a report viewer. The report viewer may also display data from the master service API (e.g., headquarter data, target data, reference data, etc.).

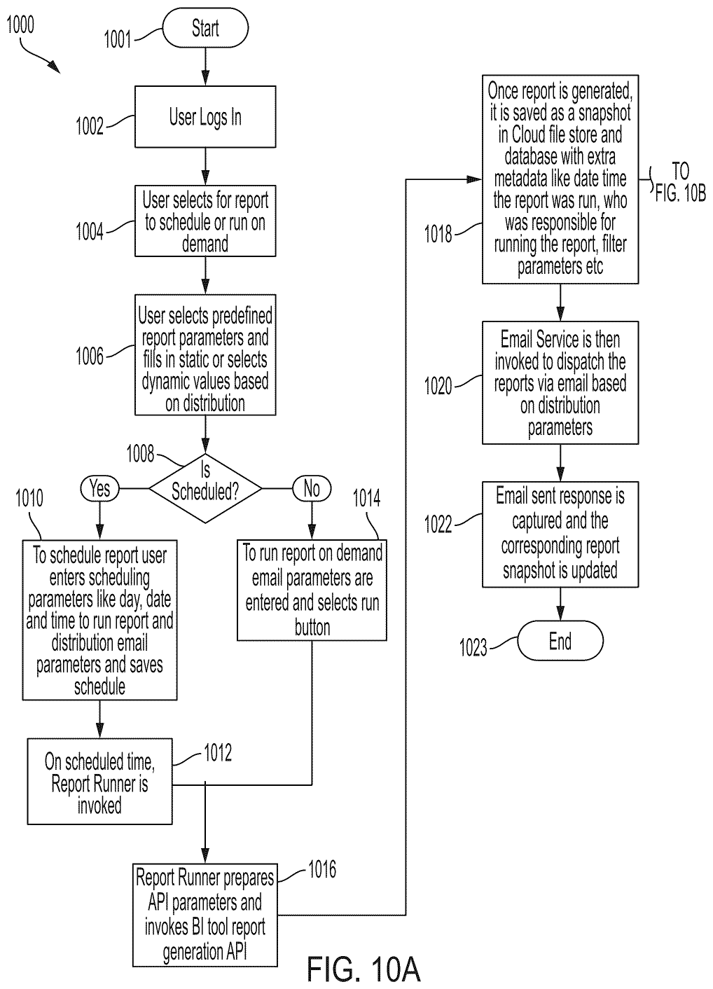

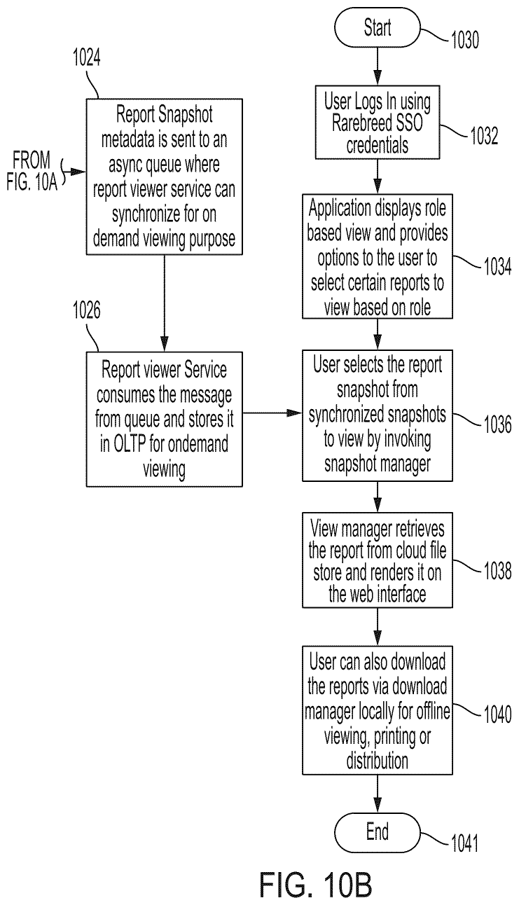

FIGS. 10A-10B shows an example flow diagram of the report distribution according to various embodiments. At block 1001, process 1000 begins. At block 1002, a user (e.g., a managing user) logs in using their credentials (e.g., a single sign-on (SSO) credential). The user, at block 1004, may then select to run a report to be scheduled to run at a specific time or run on demand. At block 1006, the user then selects predefined report parameters and fills in static values or selects dynamic values based on distribution. If the report is to be scheduled, then at 1010, the user then enters and saves scheduling parameters (e.g., day, date, and time to run report and distribution email). At 1012, the report runner is then invoked during the day, date, and/or time specified by the user. If the report is to be run on demand, at block 1014, the user inputs email parameters and then the report runner is invoked. At block 1016, the report runner prepares API parameters and invokes the business intelligence tool report generation API. At block 1018, the report is generated and a snapshot of the report is saved to the cloud file store. The snapshot is also saved in a database with metadata such as the date in which the report was run, the user responsible for running the report, filter parameters, etc. At block 1020, an email service is invoked to dispatch the report via email based on the distribution parameters. At block 1022, the email response is captured, and the corresponding report snapshot is updated. At block 1024, the process 1000 may end.

However, the process 1000 may continue to block 1026. At block 1026, the report snapshot metadata from block 1018 is sent to an asynchronous queue where the report viewer service can synchronize the report snapshot and report snapshot metadata for on demand viewing purposes. At block 1026, the report viewer service consumes the message from queue and stores it in OLTP for on demand viewing.

On demand viewing of process 1000 may start at block 1030. At block 1032, the user, if not already logged in, may log in using credentials (e.g., an SSO credential). At block 1034, an application displays views based on the role and level of the user (e.g., stakeholder or managing user). The display view may also allow the user to select certain reports to view based on the role and level of the user. At block 1036, the user selects the report snapshot from synchronized snapshots from block 1028. This may invoke the snapshot manager such that the user may view the snapshot report. At block 1038, the view manager retrieves the report from the cloud file store and renders it on the web interface. At block 1040, the user may also download the reports via download manger, locally for offline viewing, printing, or distribution. At block 1042, the on demand viewing process may end.

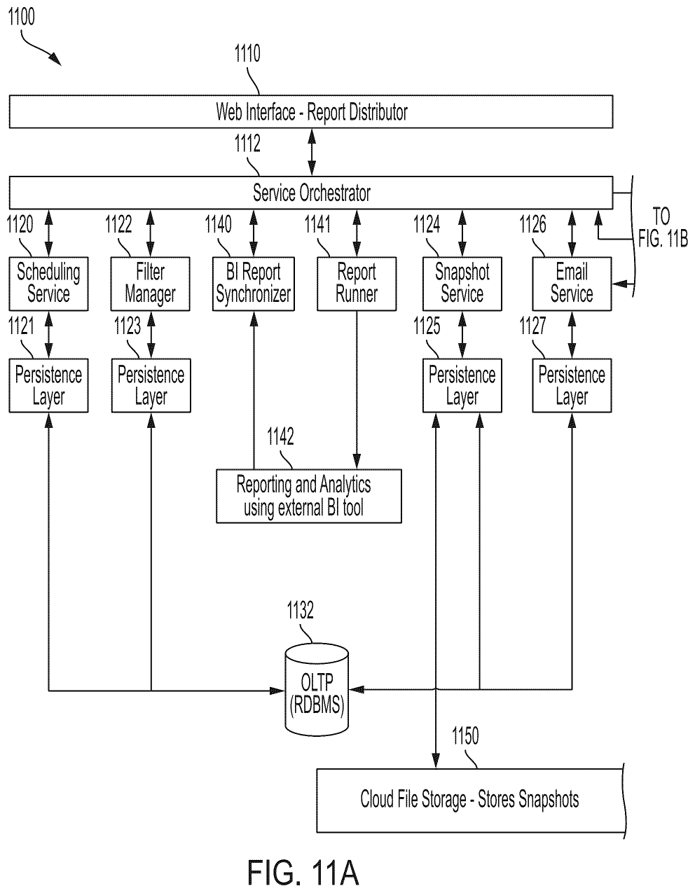

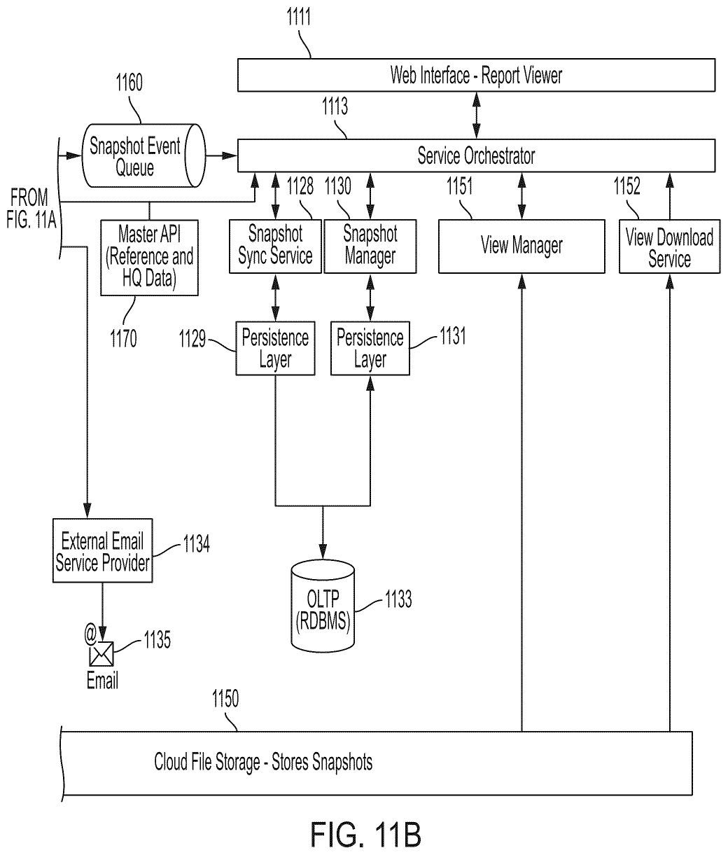

FIGS. 11A-11B is an example representation of a computer system capable of report distribution and viewing according to various embodiments. The system 1100 may include a web interface 1110 which in some instances may be a report distributor. The web interfaces 1110, 1111 may be a report distributor and/or a report viewer. The web interfaces 1110, 1111 may also allow one or more users (e.g., managing users) interface with various services. The web interfaces 1110, 1111 may communicate with the services via the service orchestrators 1112, 1113.

In some embodiments, the system 1100 may include a web interface 1110 for report distribution. The web interface 1110 may communicate with services configured to interact with a reporting and analytics engine. In some embodiments, the reporting and analytics engine may be an external business intelligence tool. In some implementations, the services include a report runner configured to interact with the reporting and analytics engine to run schedule reports or on demand reports. The reporting and analytics engine then generates a report and the business intelligence report synchronizer synchronizes the report metadata from the business intelligence tool automatically and on demand.

The system 1100 may also include various services in communication with the web interface 1110. The services may be configured to exchange data with the OLTP 1132 (e.g., RBDMS) via one or more persistence layers. For example, services may include a scheduling service 1120, a filter manager 1122, a snapshot service 1124, and an email service 1126. The scheduling service 1120 may be configured to manage the report run schedule. The filter manager 1122 may be configured to manage filter creation and association with the report. The email service 1126 may be configured to manage the distribution list for email dispatches. The email service 1126 may also be configured to request response data in the snapshot for display and future audit. The snapshot service 1124 may be configured to save a snapshot of the report and its associated metadata about the report run. In some embodiments, the snapshot service 1124 may communicate with a cloud file storage to save the snapshot of the report and the associated meta data.

The system 1100 may further include services in communication with the web interface 1111, which may be configured as a report viewer. For example, services may include a view manager and a view download service configured to communicate with the cloud file storage. The view manager may retrieve the snapshot of the report from the cloud file storage to render on the web interface. The view download service may download the report snapshot on demand.

The web interface 1111 may also communicate with services which may be configured to exchange data with an OLTP 1133. The exchange of data between the services and the OLTP may be facilitated by a persistence layer. Example services configured to exchange data with the OLTP include a snapshot sync service configured to synchronize snapshots from the report distribution service via the event queue and the snapshot manager configured to retrieve snapshots based on the filter parameters (e.g., role, hospital, region, date range, department, position, etc.).

The system 1100 may further include a snapshot event queue configured to communicate data between the service orchestrator 1112 of the report distributor web interface 1110 and the service orchestrator 1113 of the report viewer web interface 1111. The system 1100 may also include a master API which may provide reference data, target data, and headquarter data to the service orchestrators 1112, 1113.

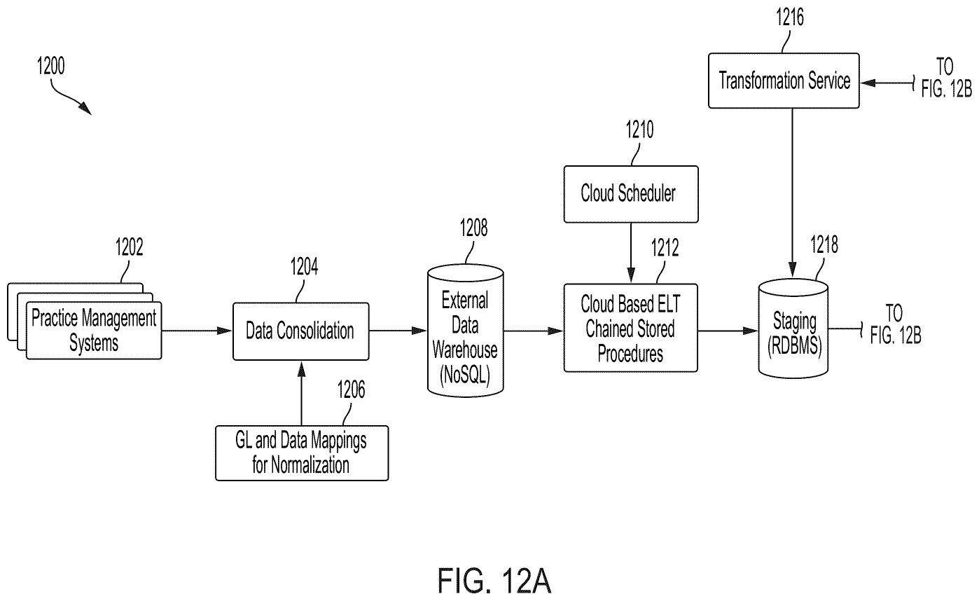

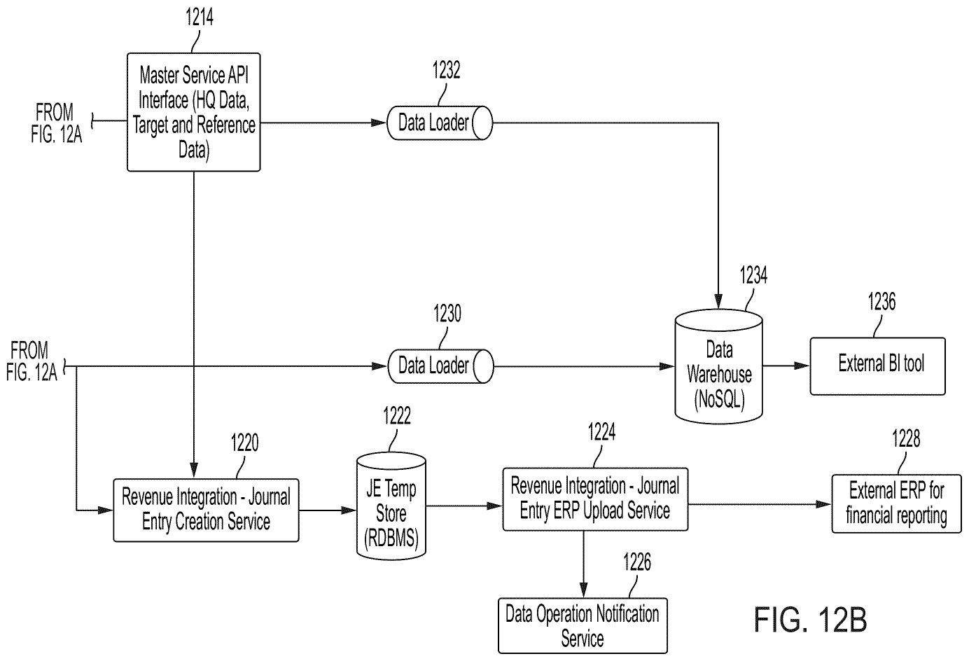

FIGS. 12A-12B is an example representation of an architecture for revenue integration according to various embodiments. The system 1200 may include a PMS 1202. Data may be extracted from the PMS 1202 on a regular cadence (e.g., daily) or on demand. Extracted data may include transaction data and/or operational data. The data consolidation 1204 may apply GL and data mapping to normalize the extracted data. In some implementations, GL categories may include total revenue, total cost of goods sold, total cost of service, total cost of goods service by position, total operating expense, other revenue, other expense, gross profit, EBITDA (earnings before interest, taxes, depreciation and amortization), net income, ownership, among other GL categories. The normalized data may then be piped to a data warehouse 1208. In some instances, the data warehouse 1208 may be a NoSQL data warehouse. Data from the data warehouse 1208 may be fed into an ELT process 1212. According to some embodiments, the ELT process may be a cloud-based ELT chained stored procedure. The ELT process may be managed by a cloud scheduler 1210. The ELT processes may include identifying changed data for each entity type and extract date ranges, logically remove data from destination for entity type based on delta, run stored procedure for entity type, staging the data temporarily for transformation, apply audit time stamps, physically remove data from destination if marked, etc.

System 1200 may also include staged data 1218. In some embodiments, data may be staged in a RDBMS. Data may be staged into key entity groups (e.g., practice, transactions, revenue categories and hierarchy, providers, client, patient, appointment, reminders, prescriptions, medical notes, etc.). Transformation services 1216 may be applied to the staged data to standardize the data to headquarter specification. Master service API 1214 may provide the transformation services 1216 with headquarter specifications transforming the staged data including headquarter data, target data, benchmark data, and/or reference data. According to some embodiments, the transformations services 1216 may add practice reference data based on headquarter practice attributes, add provider reference data based on HRIS and ERP attributes, add benchmark and target data, perform preliminary roll ups for load optimization, add audit time stamps for each operation, and/or notify data operation on each successful transformation for audit and monitoring.

Staged data may then be persisted into the data warehouse 1234. The data warehouse may be a NoSQL warehouse. The data warehouse 1234 may also be configured to store data from the master service API 1214 including headquarter data, target data, and reference data. Data from the warehouse 1234 may be fed into the business intelligence tool 1236 to generate reports, charts, decisions and actions.

Stage data may also be piped to the revenue integration 1220. The revenue integration 1220 may be a journal entry creation service that creates journal entries based on ERP specification. The journal entries generated by the revenue integration 1220 may be staged in the journal entry (JE) temporary store to be uploaded, by the journal entry ERP upload service 1224, to the ERP 1228 for financial reporting. The journal entry ERP upload service may be configured to asynchronously upload journal entries into the ERP. Once the journal entry is uploaded, audit flags and timestamps may be added to the journal entries. In some embodiments, once the journal entries are uploaded, the data operation notification service 1226 may notify the user that the journal entries have been uploaded.

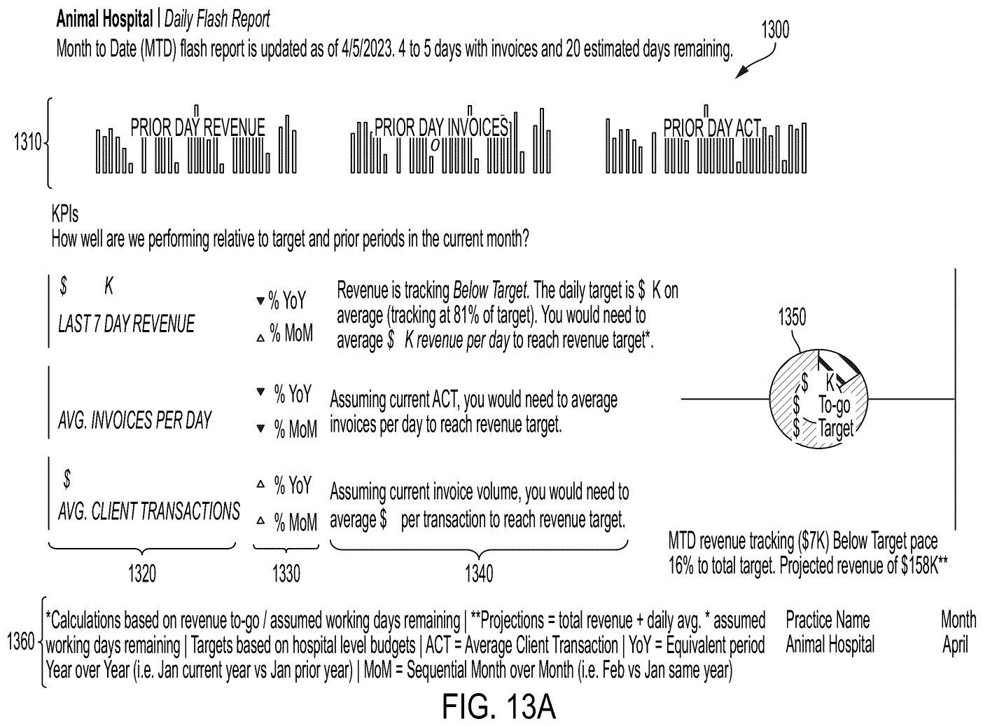

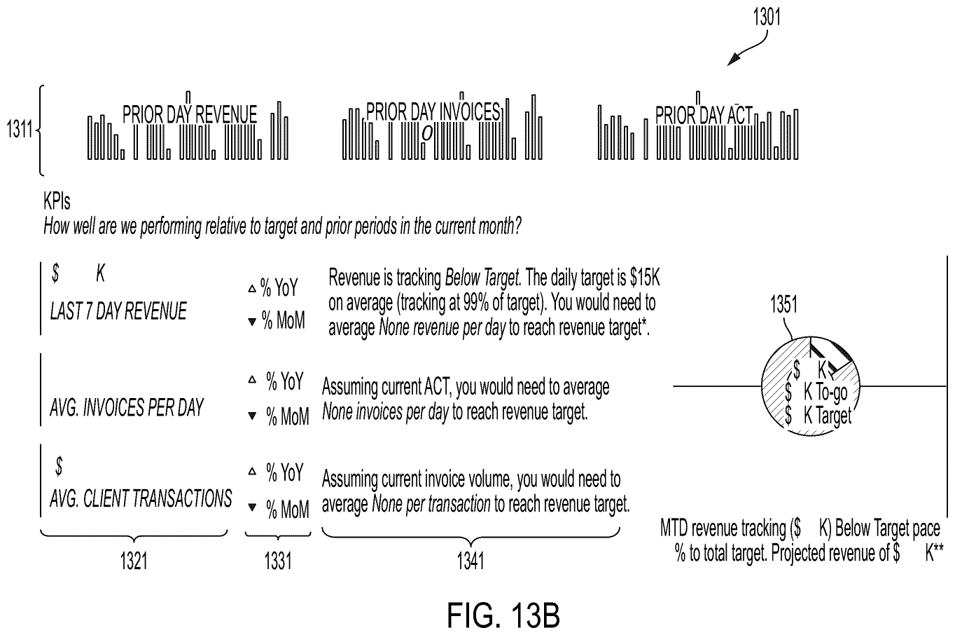

FIGS. 13-20 are example dashboard interfaces established within the system of the present invention accessible by one or more management users. The dashboards may provide users at all different levels information for improving practice and practice management. According to some embodiments, a practice performance dashboard interface may be provided that uses metrics (e.g., hundreds of metrics in various categories), the metrics when used in isolation provide a very limited understanding of the business and people, but when indexed, curated and visualized with root cause and plan then meaningful and targeted change can be implemented.

According to some embodiments, the interface may display information relating to one or more indices or metrics. The interface may display values corresponding to indices or metrics, visual representations (e.g., graphs and charts), and targets. In some embodiments, the interface may display targets. Targets may be the product of cross functional and historical data being fed into the system. Target may be incorporated into each metric as an indicator of health of practice and corporate. The information displayed by the interface may allow stakeholders to determine a corrective plan, if necessary.

FIG. 13A shows an example interface 1300 configured to display a daily flash report for the practice according to various embodiments. In some embodiments, the flash report may display at least one value corresponding the at least one metric 1320. In some embodiments, the metrics may be a key performance indicator. The flash report may include various metrics 1320 such as the last seven day revenue, average invoices per day, average client transactions (ACT), among a number of other metrics. The flash report may also include a visual representation of the metrics over time 1310. For instance, the visual representation may be a graph or bar chart of the metrics over a period of time, such as revenue, invoices, or ACT. In some embodiments, the graph or bar chart may include a value corresponding to the prior day's metrics, such as the prior day's revenue, invoices, or ACT. The flash report may also include other metrics 1330 such as the year-over-year (YoY) or month-over-month (MoM) which may correspond to the metrics of 1320. The flash report may also include root cause information 1340 for the metrics of 1320 for performance measures along a suggested plan. Such cause information and plan information may be abstracted from previous engagements with other practices and may be applied automatically to the system being evaluated. The root cause information may include suggestions for reaching a target or may indicate if performance is below a target. In some embodiments, the daily flash report may include a visual representation 1350 of a target and the progress made in relation to the target. In some embodiments, the flash report may include a legend and/or notes 1360 relating to the flash report. The legend 1360 may include information such as description of the metrics and corresponding calculations, practice name of the practice being analyzed, among others.

FIG. 13B shows another example interface 1301 configured to display a daily flash report for the practice according to various embodiments. In some embodiments, interface 1301 may be similar to interface 1300 of FIG. 13A. The flash report may information relating to various metrics (e.g., last seven day revenue, average invoices per day, average client transactions, etc.). The information relating to the metrics may include visual representations 1311 of the metrics over time, values 1321 corresponding to the metrics, root cause information 1341 for the metrics. The flash report may also include other metrics 1331 such as the YoY or MoM which may correspond to the metrics. The flash report may further include a visual representation 1351 of a target and the progress made in relation to the target.

FIGS. 14A-1 and 14A-2 shows an example interface 1400 configured to display a weekly flash report for the practice according to various embodiments. In some embodiments the flash report may display a value or visual representation corresponding to at least one metric. For example, the flash report may display a graph or bar chart 1410 of the revenue, invoices, or ACT over time. The chart or bar graph may also display the prior week's metrics, such as the prior week's revenue, invoices, or ACT. The flash report may also display a visual representation describing the progress towards a target for a particular metric. For example, pie charts 1420 may be displayed that show the progress towards goals for the monthly, quarterly, and yearly revenue target. The display 1430 may also describe progress towards the target, projected metric based on the current value associated with the metric, and the difference or change in the metric compared to previous recorded metrics (e.g., % increase versus last year MTD). In some embodiments, the flash report may also display a chart 1440 of the metric over time. For example, the chart may represent the revenue per month over the span of a year for the current year and the previous years. In some embodiments, the flash report may display a table 1450 including metrics. The table may include evaluations of the metrics per week. The table may further include the percentage of the goal encapsulated by the revenue for the week and the YoY percentage change. The flash report may include a legend and/or notes 1460 relating to the flash report. The legend 1460 may include information such as description of the metrics and corresponding calculations, practice name of the practice being analyzed, among others.

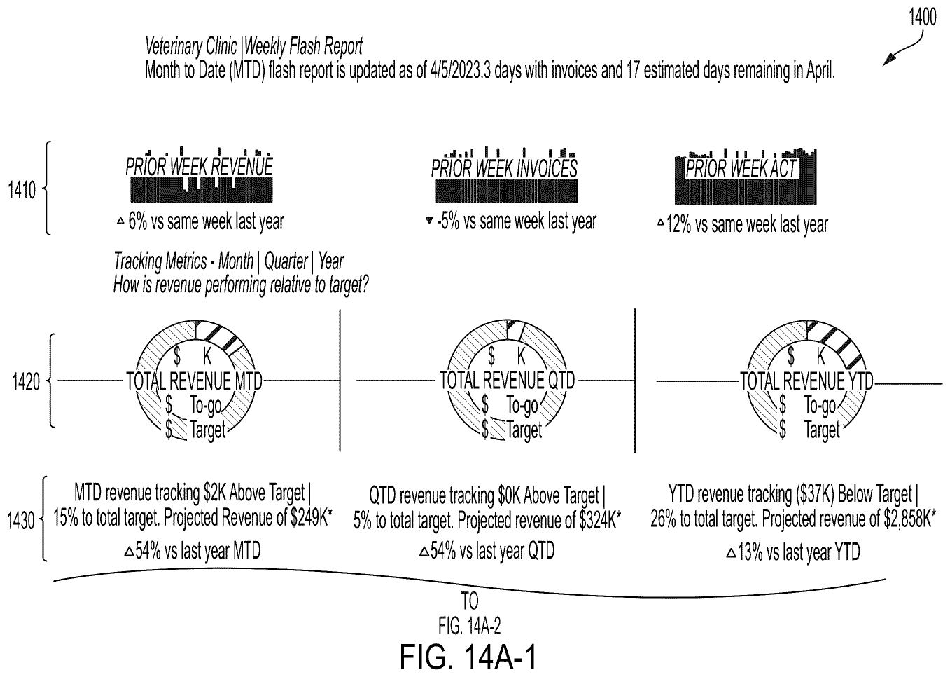

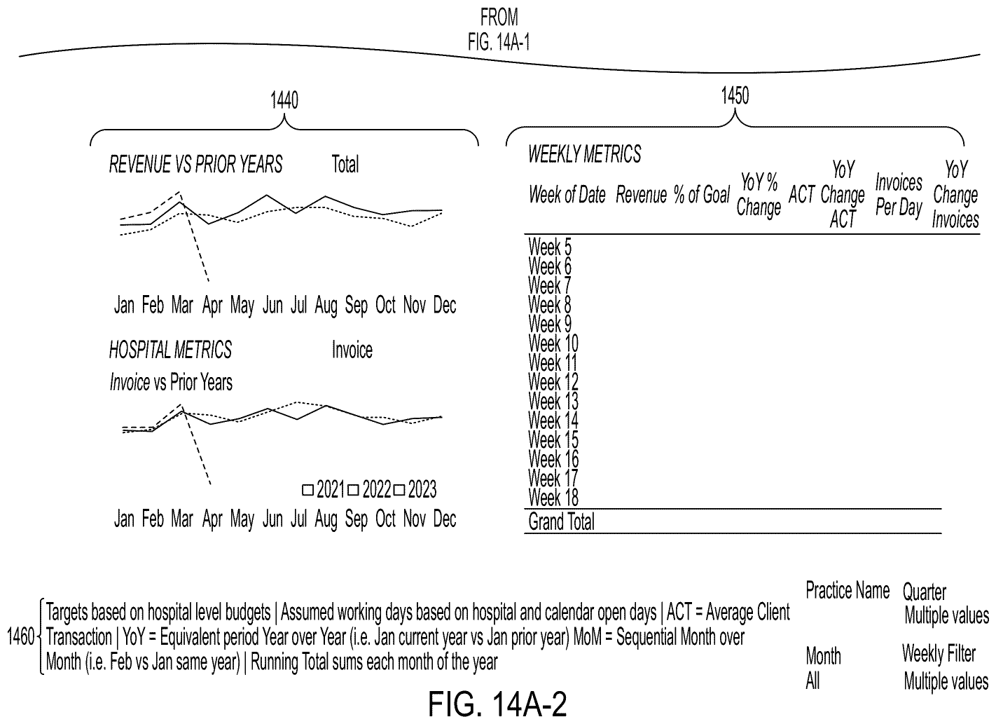

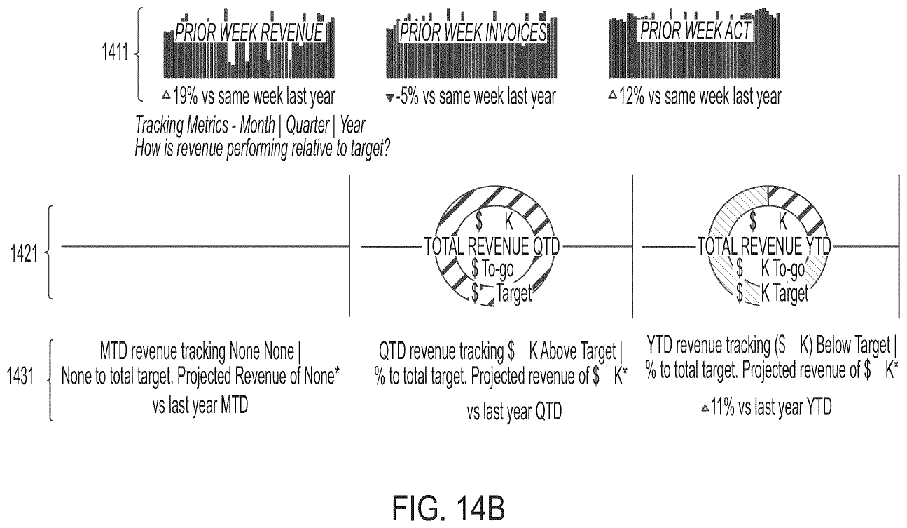

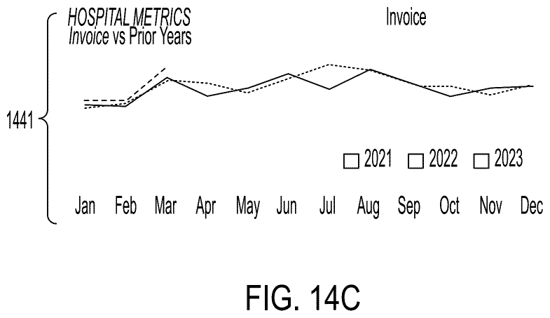

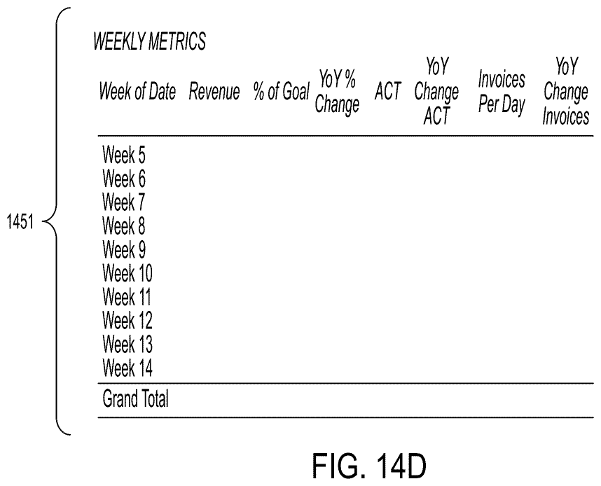

FIGS. 14B-14D show example interfaces configured to display visual representations of metrics according to various embodiments. The visual representation may include a graph or bar chart 1411 of the revenue, invoices, or ACT over time. The chart or bar graph may also display the prior week's metrics, such as the prior week's revenue, invoices, or ACT. In some embodiments, the visual representation may describe the progress towards a target for a particular metric. For example, pie charts 1421 may be displayed that show the progress towards goals for the monthly, quarterly, and yearly revenue target. Pie chart 1421 may have a corresponding description 1431 of the progress towards the target, projected metric based on the current value associated with the metric, and the difference or change in the metric compared to previous recorded metrics (e.g., % increase versus last year MTD). The visual representation may further include a chart 1441 of the metric over time. For example, the chart may represent the revenue per month over the span of a year for the current year and the previous years. In some embodiments, the flash report may display a table 1451 including metrics. The table may further include the percentage of the goal encapsulated by the revenue for the week and the YoY percentage change.

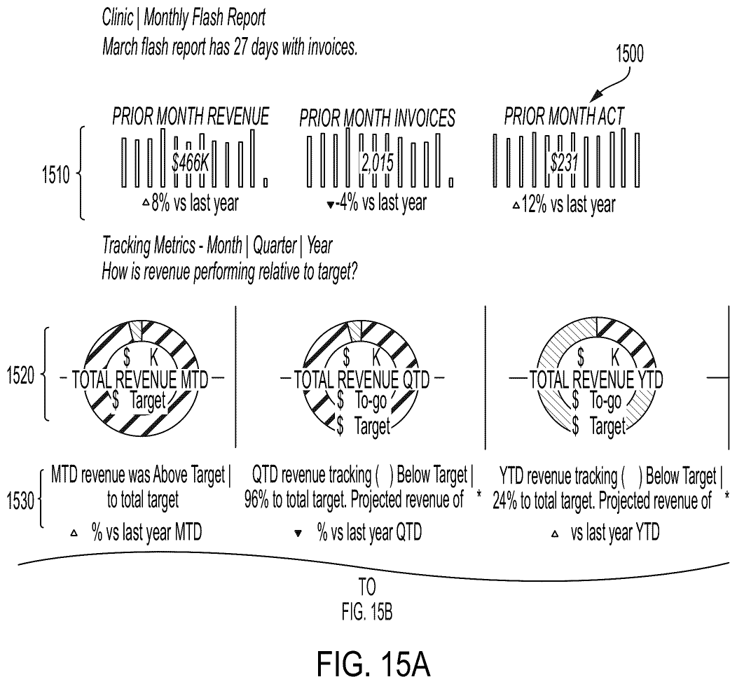

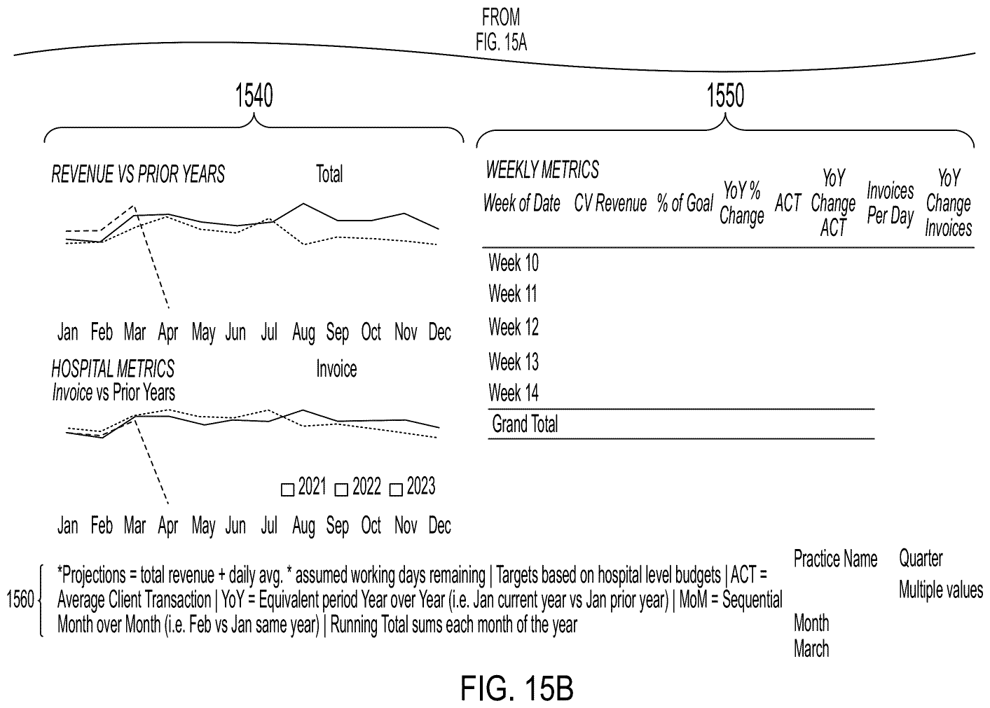

FIGS. 15A-15B shows an example interface 1500 configured to display a monthly flash report for the practice according to various embodiments. In some embodiments, the display of FIGS. 15A-15B may be similar to the display of FIGS. 14A-1 and 14A-2. In some embodiments, the flash report may display a value or visual representation corresponding to a metric. For example, the flash report may display a graph or bar chart 1510 of the revenue, invoices, or ACT over time. The chart or bar graph may also display the prior month's metrics, such as the prior month's revenue, invoices, or ACT. The flash report may also display a visual representation describing the progress towards a target for a particular metric. For example, pie charts 1520 may be displayed that show the progress towards goals for the monthly, quarterly, and yearly revenue target. The display 1530 may also describe progress towards the target, projected metric based on the current value associated with the metric, and the difference or change in the metric compared to previous recorded metrics (e.g., % increase versus last year MTD). In some embodiments, the flash report may also display a chart 1540 of the metric over time. For example, the chart may represent the revenue per month over the span of a year for the current year and the previous years. In some embodiments, the flash report may display a table 1550 including metrics. The table may include evaluations of the metrics per week. The table may further include the percentage of the goal encapsulated by the revenue for the week and the YoY percentage change. The flash report may include a legend and/or notes 1560 relating to the flash report. The legend 1560 may include information such as description of the metrics and corresponding calculations, practice name of the practice being analyzed, among others.

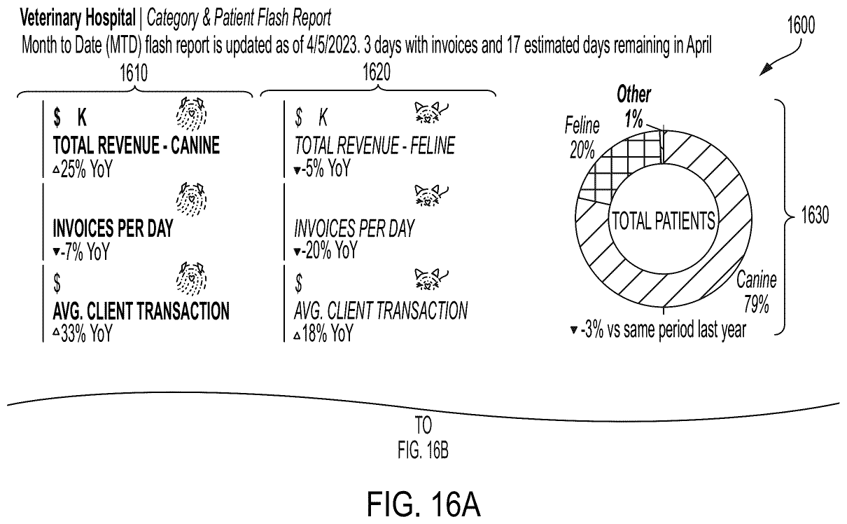

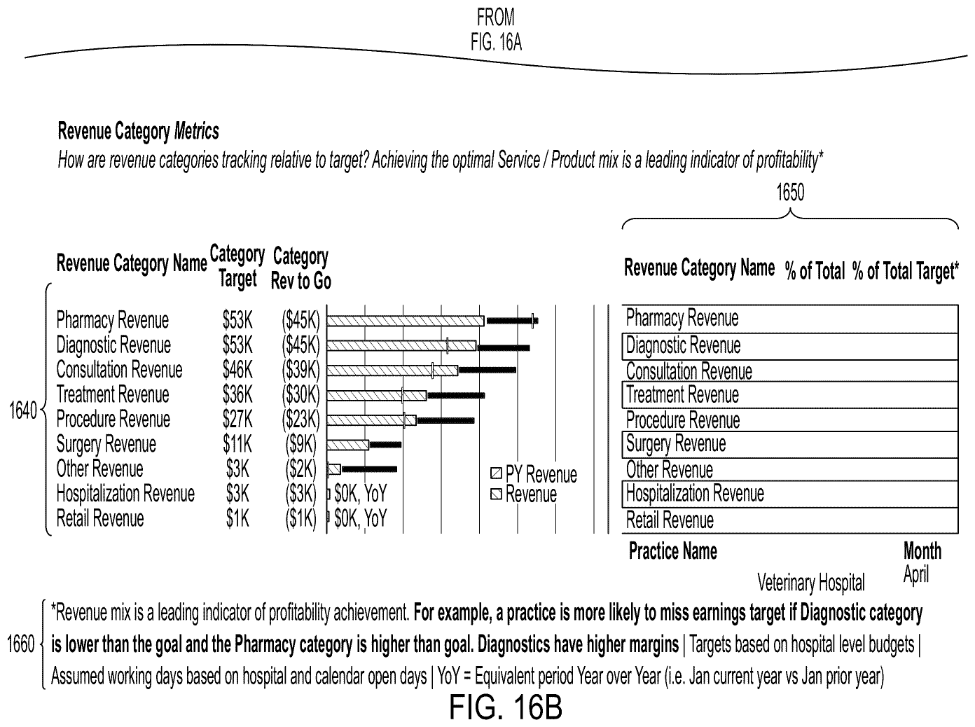

FIGS. 16A-16B shows an example interface 1600 configured to display a category and patient flash report for the practice according to various embodiments. In some embodiments, the flash report may display at least one metric for at least one category of patient. For instance, the display may show at least one metric for canines 1610 and may show at least one metric for felines 1620. The metrics may include total revenue, invoices per day, and ACT. The display may also show the YoY percentage change for each metric for each category of patient. In some embodiments, the flash report may also display a visual 1630 for understanding the different categories of patients and the percentage each category of patient comprises of the total. For instance, the visual may be a pie chart displaying the percentage composition of feline, canine, and other patients that comprise of the patient population for the practice. The flash report may also display values and charts 1640 of the categories that comprise a metric. For example, the flash report may display revenue category metrics such as pharmacy revenue, diagnostic revenue, consultation revenue, treatment revenue, procedure revenue, surgy revenue, hospital revenue, retail revenue, and other revenue. For each category, the flash report may also display 1650 the percentage of the total revenue that each category comprises and the percentage of the total target that each category comprises. The flash report may include a legend and/or notes 1660 relating to the flash report. The legend 1660 may include information such as description of the metrics and corresponding calculations, practice name of the practice being analyzed, among others.

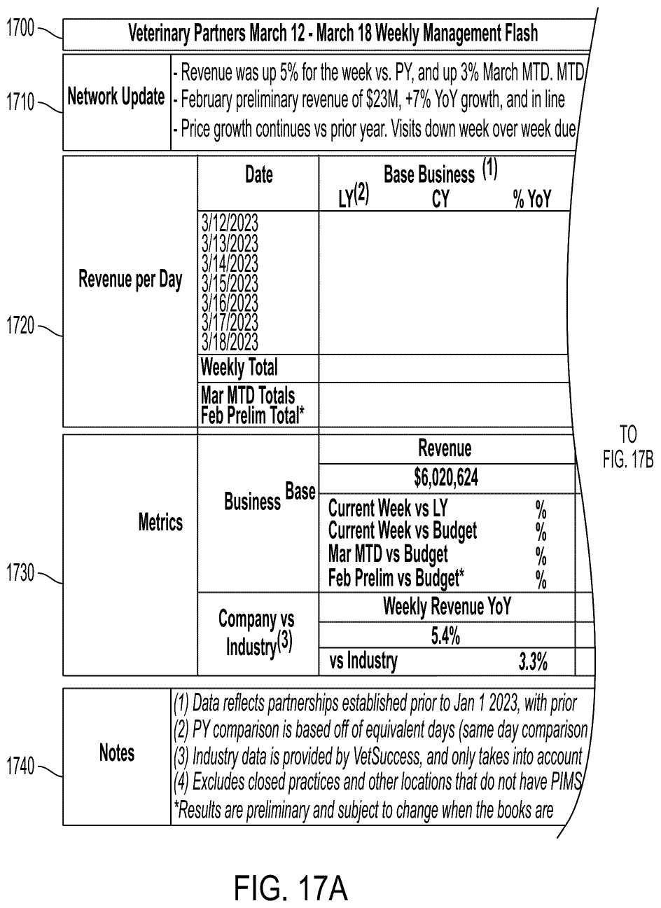

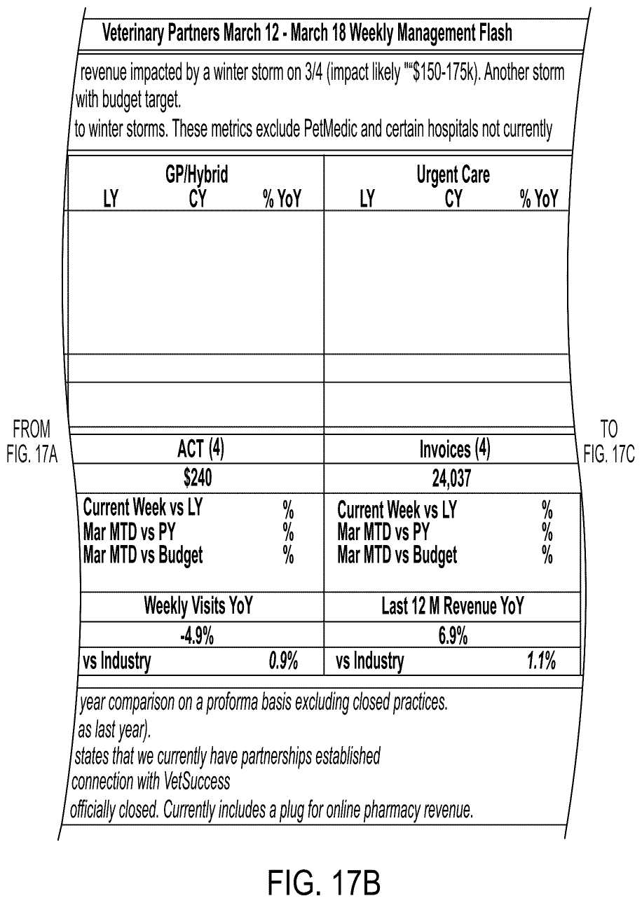

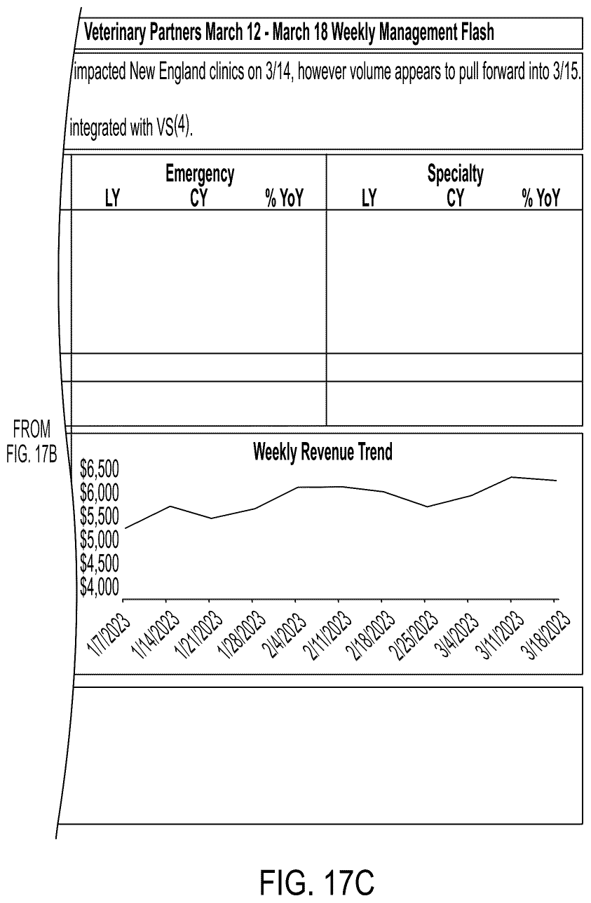

FIGS. 17A-17C shows an example interface 1700 configured to display a weekly management flash report for various practice according to various embodiments. In some embodiments the flash report may display 1710 analyses and root causes relating to metrics. The flash report may also include the metrics evaluated on a daily basis 1720. For instance, the flash report may display the revenue per day. The metrics may be further broken down into categories. In this exemplary flash report, the revenue per day may be broken down into base business, GP/hybrid, urgent care, emergency, and specialty. The flash report may also include evaluations of other metrics such as ACT and invoices and may display the weekly trend via a chart or graph 1730. The flash report may include a legend and/or notes 1740 relating to the flash report. The legend 1740 may include information such as description of the metrics and corresponding calculations, practice name of the practice being analyzed, among others.

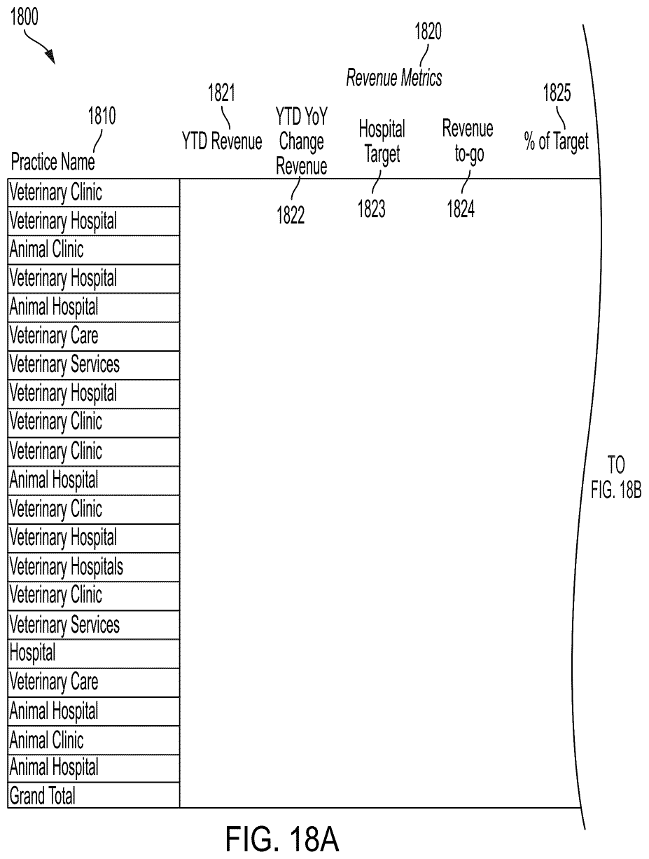

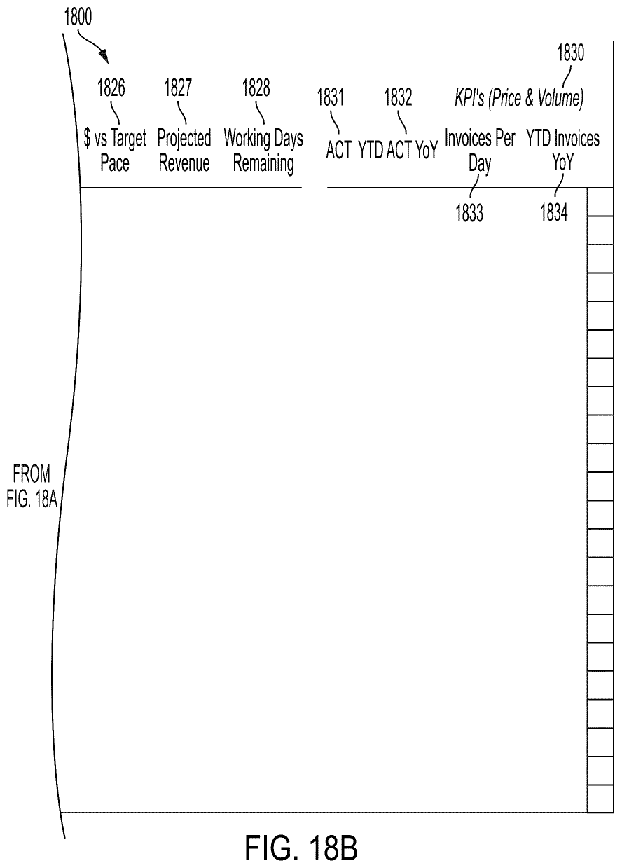

FIGS. 18A-18B shows an example interface configured to display a table 1800 including metrics and information relating to the metrics associated with individual practices 1810 according to various embodiments. Table 1800 may include information relating to metrics such as revenue, price, and volume. For example, information relating to the revenue metric 1820 may include year to date (YTD) revenue 1821, YTD YoY change revenue 1822, hospital target 1823 (e.g., practice target revenue), revenue to-go 1824 (e.g., amount of revenue remaining to meet target), percentage of target 1825 (e.g., percentage of target achieved), dollars versus target pace 1826, projected revenue 1827, and working days remaining 1828. In some embodiments, table 1800 may include KPIs, which may include information relating to one or more metrics. The KPI 1830 of table 1800 may include information relating to price and volume. Information relating to KPI 1830 may include ACT 1831, YTD ACT YoY 1832, invoices per day 1833, and YTD Invoices YoY 1834.

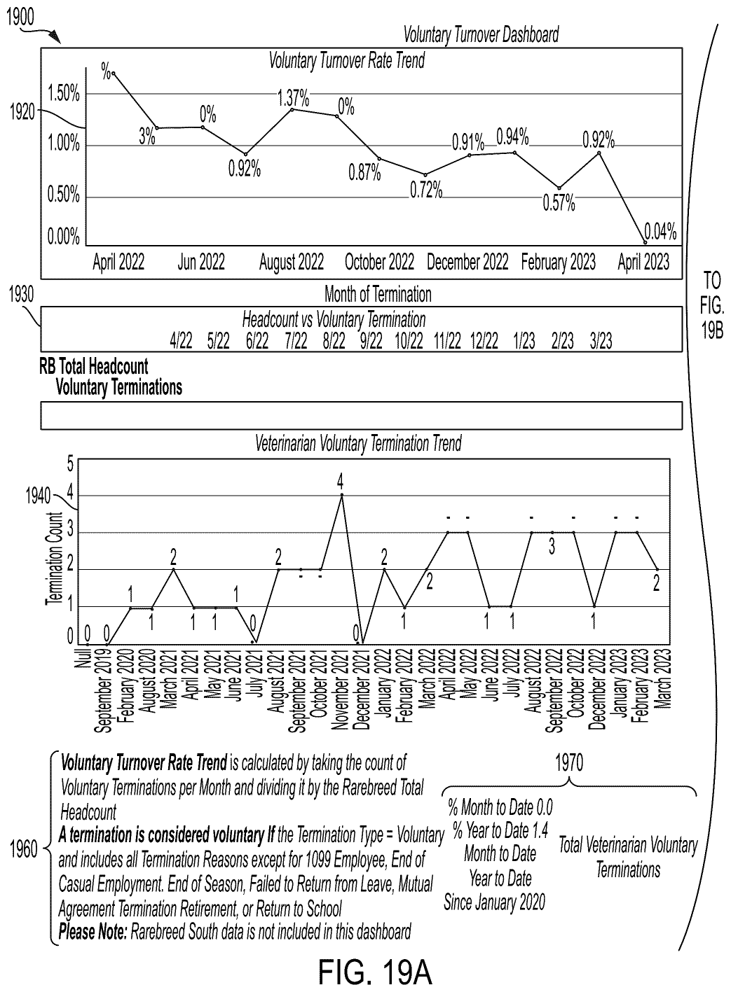

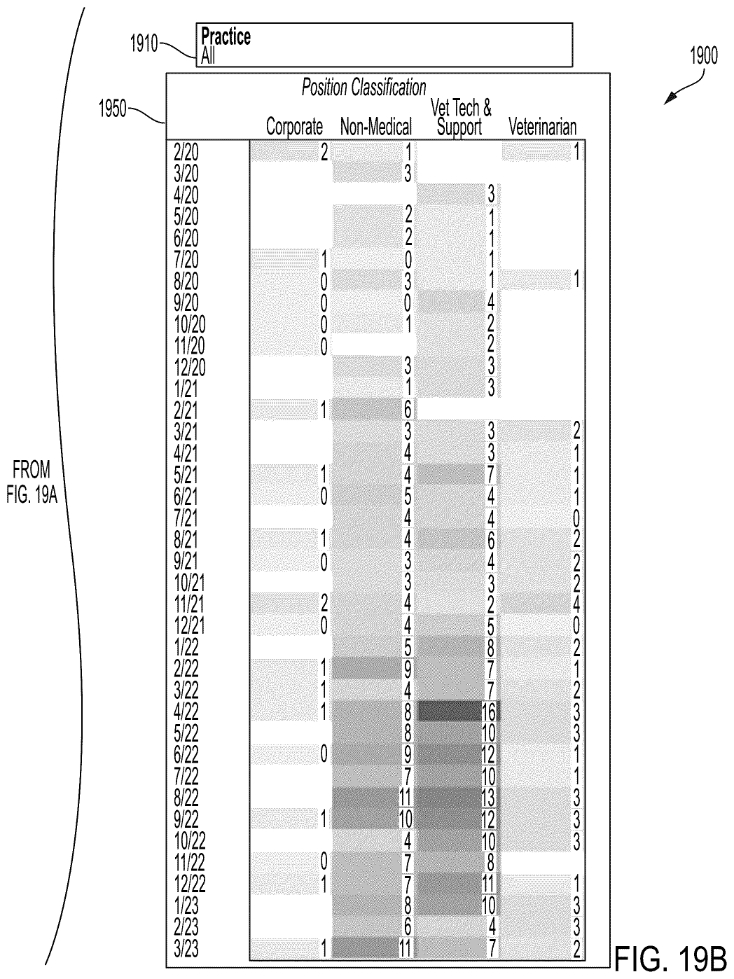

FIGS. 19A-19B shows an example interface 1900 configured to display a dashboard relating to a metric according to various embodiments. For example, the interface 1900 may display a voluntary turnover dashboard. In some embodiments, the dashboard may display which practices are being evaluated 1910. The dashboard may also include various visual representations relating to metric including (e.g., scatter plots, tables, etc.). In some embodiments, scatter plots may be used to illustrate a metric over time. For instance, scatter plot 1920 may show voluntary turnover rate over time and scatter plot 1940 may show veterinarian voluntary termination over time. In some embodiments, tables such as tables 1910 and 1930 may visually represent information relating to metrics. Table 1910 may depict the voluntary termination per position over time and table 1930 may depict the headcount versus voluntary termination over time. The dashboard may also display a legend and/or notes 1960 relating to the flash report. The legend 1960 may include information such as description of the metrics and corresponding calculations, practice name of the practice being analyzed, among others. The dashboard may further display further metrics 1970 including total veterinarian voluntary terminations, percent month to date voluntary terminations, percent year to date voluntary terminations, month to date voluntary terminations, year to date voluntary terminations, and voluntary terminations since a particular date.

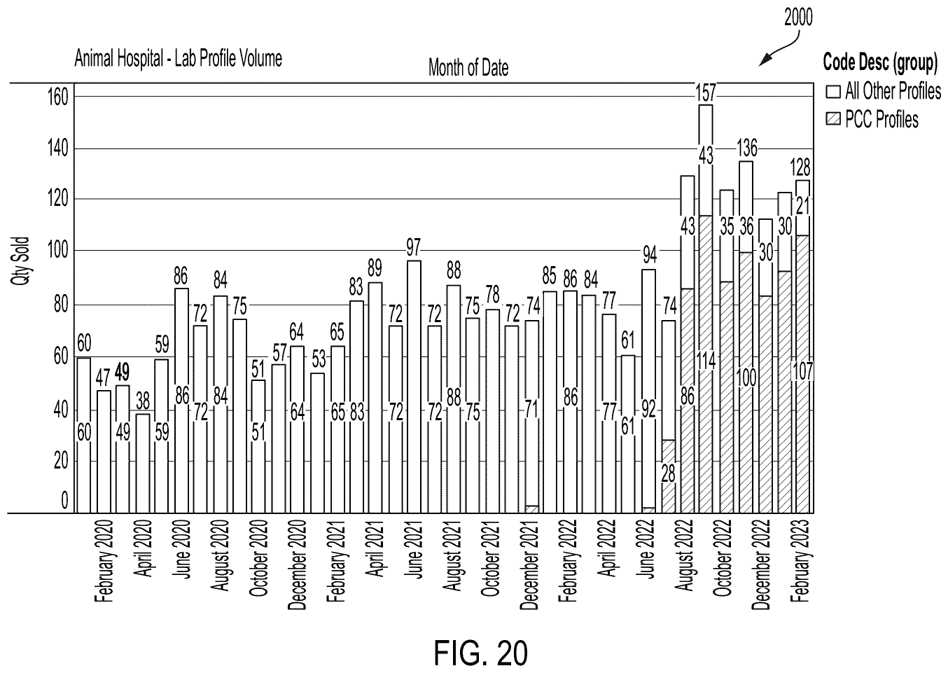

FIG. 20 shows an example interface configured to display a metric over time according to various embodiments. For example, the interface may be configured to display a bar chart 2000 of the quantity of lab profile volume sold over time (e.g., monthly, yearly, etc.). In some embodiment, the bar chart may include the lab profile volume for a particular practice and all other practices to allow for comparison of the performance of the particular practice in relation to other practices. Comparison of historical data on a practice's metrics or indices with other practices may aid in determining root causes and plans.

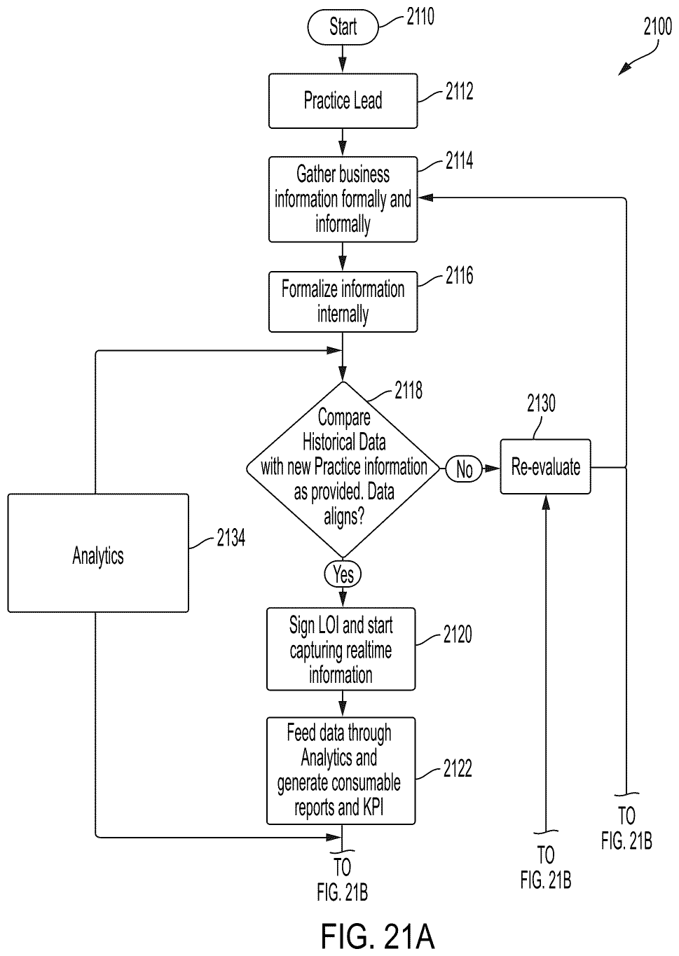

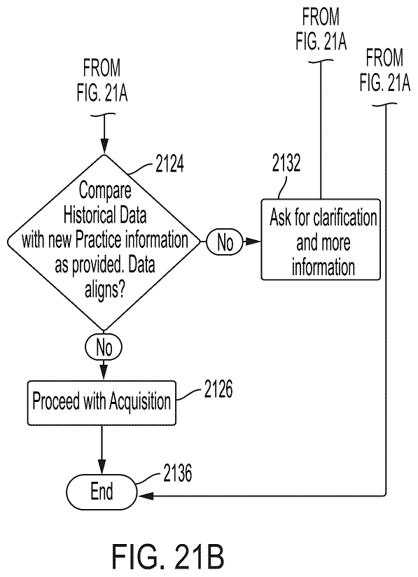

FIGS. 21A-21B shows an example process for acquisition of a new practice according to various embodiments. At block 2110, process 2100 begins.

At block 2112, leads on practices to acquire may be obtained. At block 2114, business information about the new, potential practice to be acquired may be obtained formally or informally. Business information may include personnel, infrastructure, financial, and marketing information, among other business information. In some embodiments, the business information may be formal or informal information. At block 2116, the business information gathered may be formalized internally. In some embodiments, the information gathered may be formalized to be used within an ecosystem (e.g., ecosystem 100) which may be implemented across disparate SAAS software.

At block 2118, the historical data may be compared with new practice information provided. In some embodiments, the new practice information may be compared against benchmarks. Benchmarks may be based on market specific data and internal analytics generated by other practices, among other data. In some embodiments, the benchmarks for the new practice may be based on the size of the practice, volume, and region of the practice to be acquired. The new practice information may be compared with one or more historical data/benchmarks. The historical data/benchmarks may be based on baseline revenue and profitability, Region specific Client Base and Retention, Patient visits, appointment times and fill rates, Average Transaction (e.g., average revenue generated per patient visit), Services offered, Doctors, Doctor vs Staff ratio and efficiencies, Number of Exam Rooms, Number of Exams, Inventory turnover, Practice NPS via client online reviews, testimonials, and social media reputation, or Local market dynamics.

If the new practice information does not align with the historical data/benchmarks, then at block 2130 the potential practice to be acquired may be re-evaluated. In some embodiments, after re-evaluating a practice, the process may continue to block 2114 such that further business information may be gathered. In other embodiments, after re-evaluating a practice, the process may continue to block 2136 such that the new practice is not acquired.

In some embodiments, if the new practice information does align with the historical data/benchmarks, then at block 2120, a letter of intent (LOI) may be signed and real-time information about the new practice may be captured. At block 2122, data from the new practice may be fed through the analytics 2134 to generate consumable reports and KPIs. In some embodiments, the analytics 2134 may verify the authenticity of the business information gathered at 2114. If discrepancies in the information gathered at 2114 and the analytics 2134 is detected, then clarification and more information can be obtained, or the acquisition may not proceed. If little to no discrepancies are detected, then the acquisition may proceed.

At block 2124, historical data/benchmark data may be compared with the new practice information. If the new practice information does not align with the historical data/benchmark data, then clarification and more information may be asked at block 2132. If the new practice information does not align with the historical data/benchmarks, then at block 2132 the potential practice to be acquired may be asked to provide clarification and more information. The process may then proceed to block 2130 such that the potential practice to be acquired may be re-evaluated. In some embodiments, after re-evaluating a practice, the process may continue to block 2114 such that further business information may be gathered. In other embodiments, after re-evaluating a practice, the process may continue to block 2136 such that the new practice is not acquired.

In some embodiments, if the new practice information does align with the historical data/benchmarks, then at block 2126, the acquisition may proceed. At block 2136, process 2100 may end.

### Example Computer System

Aspects according to some embodiments relate to a system including one or more computer programs configured to enable a user to input data, transfer data, collate data, analyze data, and/or transfer data analyses, all related to the content and management of a group of practices. The system of the present invention is a set of functions embodied in the one or more computer program for executing primary actions associated with the methods to be described herein.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be understood that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be understood that one implementation of the embodiments of the present invention comprises at least one non-transitory computer-readable storage medium (e.g., a computer memory, a portable memory, a compact disk, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be understood that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

What is claimed is:

1. A distributed computer system comprising:
a component configured to extract practice management data from one or more practice management data sources relating to a practice being evaluated;
a component configured to map the extracted practice management data into at least one normalized data set;
a component configured to process and store transactional data from the extracted practice management data; and
a component adapted to analyze the normalized data set and transactional data; and responsive to an analysis operation, determine a performance measure of the practice being evaluated.

2. The distributed computer system according to claim 1, wherein the one or more practice management data sources relating to the practice being evaluated comprise one or more of a group including:
financial information;
human resource information;
patient information; and
medical information.

3. The distributed computer system according to claim 2, wherein the component adapted to analyze the normalized data set and transactional data; and responsive to an analysis operation, determine the performance measure of the practice being evaluated determines a performance measure related to at least one of a group comprising:
financial performance;
human resource performance;
patient performance; and
medical performance.

4. The distributed computer system according to claim 3, further comprising an interface component configured to output the performance measure related to at least one of the group comprising financial performance, human resource performance, patient performance, and medical performance to at least one user type or computing entity.

5. The distributed computer system according to claim 4, wherein the at least one user type comprises at least one of a group of user types including:
a practice management user;
an outside evaluator user evaluating the practice being evaluated; and
an employee user of the practice being evaluated.

6. The distributed computer system according to claim 1, further comprising a plurality of components that interface with existing ones or the one or more practice management data sources.

7. The distributed computer system according to claim 1, wherein the performance measure of the practice being evaluated further comprises at least one of a group including:
an employee experience index;
a client experience index;
a financial index;
a labor index;
an inventory index;
a volume index; and
a revenue index.

8. The distributed computer system according to claim 1, wherein the performance measure of the practice being evaluated further comprises at least one of a group including root cause, plan information, and progress toward a target.

9. The distributed computer system according to claim 1 further comprising a component configured to determine a performance measure across multiple practices.

10. The distributed computer system according to claim 1 further comprising a component configured to display the performance measure of the practice.

11. The distributed computer system according to claim 10 wherein the displayed performance measure may be visualized using at least one of a group including a chart, plot, or table.

12. A method for managing practice data comprising:
extracting practice management data from one or more practice management data sources relating to a practice being evaluated;
mapping the extracted practice management data into at least one normalized data set;
processing and storing transactional data from the extracted practice management data; and
analyzing the normalized data set and transactional data; and responsive to an analysis operation, determining a performance measure of the practice being evaluated.

13. The method according to claim 12, wherein the one or more practice management data sources relating to the practice being evaluated comprise one or more of a group including:
financial information;
human resource information;
patient information; and
medical information.

14. The method according to claim 13, wherein the act of analyzing the normalized data set and transactional data; and responsive to an analysis operation, determining the performance measure of the practice being evaluated includes determining a performance measure related to at least one of a group comprising:
financial performance;
human resource performance;
patient performance; and
medical performance.

15. The method according to claim 14, further comprising outputting, by an interface, the performance measure related to at least one of the group comprising financial performance, human resource performance, patient performance, and medical performance to at least one user type or computing entity.

16. The method according to claim 15, wherein the at least one user type comprises at least one of a group of user types including:
a practice management user;
an outside evaluator user evaluating the practice being evaluated; and
an employee user of the practice being evaluated.

17. The method according to claim 11, further comprising an act of interfacing with existing ones or the one or more practice management data sources.

18. The method according to claim 11, wherein the performance measure of the practice being evaluated further comprises at least one of a group including:
an employee experience index;
a client experience index;
a financial index;
a labor index;
an inventory index;
a volume index; and
a revenue index.

19. The method according to claim 11, wherein the performance measure of the practice being evaluated further comprises at least one of a group including root cause, plan information, and a progress toward a target.

20. The method according to claim 11 further comprising a component configured to display the performance measure of the practice, wherein the displayed performance measure may be visualized using at least one of a group including a chart, plot, or table.
